(19) United States
(12) Patent Application Publication
(10) Pub. No.: US 2006/0078498 A1
(43) Pub. Date: Apr. 13, 2006

(54) Methods for the identification of novel ligands for the g protein-coupled receptor (gpcr) 192

(75) Inventors: Thomas Buckholz, Milford, CT (US); Mark Vandenberg, Wallingford, CT (US); Carla Prllrgrino, Madison, CT (US); Stefan Heitmeier, Wuelfrath (DE); Ian Taylor, Madison, CT (US); Richard Gedrich, Guilford, CT (US)

Correspondence Address: JEFFREY M. GREENMAN, BAYER PHARMACEUTICALS CORPORATION, 400 MORGAN LANE, WEST HAVEN, CT, 06516, US

(73) Assignee: Bayer Pharmaceuticals Corporatioh, West Haven, CT (US)

(21) Appl. No.: 10/504,588

(22) PCT Filed: Mar. 28, 2003
(86) PCT No.: PCT/US03/09522

§ 371 Date: Jun. 6, 2005

Related U.S. Application Data
(60) Provisional application No. 60/368,849, filed on Mar. 28, 2002.

(51) Int. Cl. A61K 49/00; C12Q 1/68; G01N 33/567; A61K 39/395; A61K 48/00

(52) U.S. Cl. 424/9.2; 435/6; 435/7.2; 424/144.1; 514/44

(57) ABSTRACT

The present invention relates to methods for the identification and characterization of polypeptides with ligand activity for the G protein coupled receptor (GPCR) 192. The invention encompasses the use of the ligands in combination with the receptor for the development of assays/kits for the identification of molecules that affect the ability of the ligand to interact with the receptor. In addition, the invention relates to molecules affecting the expression of the ligands (e.g., antisense DNA, ribozymes, antibodies) that may modulate the activity of the receptor. The invention also encompasses the use of the ligands and their derivatives, molecules affecting ligand expression, and compounds that modulate receptor activity for the treatment of disorders involving GPCR 192, such as those of the central nervous system, metabolic disorders (e.g., pancreatic disorders), gastrointestinal disorders, immune disorders, and cancer.

This application claims benefit of U.S. Provisional Application Ser. No. 60/368,849, filed on Mar. 28, 2002, the contents of which are incorporated herein by reference in their entirety.

### FIELD OF THE INVENTION

The present invention relates to methods for the identification and characterization of ligands for the G protein coupled receptor (GPCR) 192. The invention also encompasses the use of the ligands in combination with the receptor for the development of assays/kits for the identification of molecules that affect the ability of the ligand to interact with the receptor, molecules affecting the expression of the ligands that may modulate the activity of the receptor, and the use of these ligands and their derivatives for the treatment of disorders involving GPCR 192

### BACKGROUND OF THE INVENTION

Many important biological processes are mediated by signal transduction pathways involving G proteins. The G protein-coupled receptor (GPCR) superfamily includes receptors for hormones, neurotransmitters, growth factors, and viruses. Ligands for GPCRs include a wide variety of diverse agents such as protein hormones, peptides, chemokines, lipids, biogenic amines, divalent cations, and proteases (Ji et al., J. Biol. Chem. 273:17299-17302, 1998). These receptors also play an important role in sensory perception (e.g., vision and smell). GPCRs are expressed in a wide variety of cell and tissue types. This, along with the diverse array of ligands, indicates that GPCRs play roles in a wide variety of physiological responses. Therefore, it is likely that they also play a role in a number of pathologies.

GPCRs (also known as 7TM receptors) have been characterized as including seven conserved hydrophobic stretches of about 20 to 30 amino acids that are postulated to span the cell membrane. The hydrophobic membrane spanning regions are connected by hydrophilic loops. GPCRs have single conserved cysteine residues in each of the first two extracellular loops, which form disulfide bonds that are believed to stabilize functional protein structure (Strader et al., Ann. Rev. Biochem. 63:101-132, 1994).

GPCRs are transmembrane proteins that transduce signals across the cell membrane, initiating a second messenger response within the cell. GPCRs are coupled inside the cell by heterotrimeric G proteins to various intracellular enzymes, ion channels, and transporters (Stadel et al., Trends Pharmacol. Sci., 18:430-437, 1997). Different G protein alpha-subunits preferentially stimulate particular effectors to modulate various biological functions in a cell. Phosphorylation of cytoplasmic residues of GPCRs is also an important mechanism for the regulation of some GPCRs.

Currently, numerous GPCRs exist for which a ligand, endogenous or synthetic, is not known, that is, the receptors are “orphans.” Ligands for these receptors are useful since they aid in determining the function(s) of the orphan recptors. In addition, identification of the physiologically relevant ligands for a GPCR enables the design of assays that facilitate the identification of compounds that can alter the function of the receptor. Compounds that alter receptor function may be used to treat diseases involving the receptor.

### SUMMARY OF THE INVENTION

The present invention relates to the identification and characterization of ligands, prokineticin 1 (PROK1) and prokineticin 2 (PROK2), for the orphan GPCR 192. The present invention also relates to the use of the ligands or biologically active derivatives of the ligand (e.g., mutant proteins, peptide fragments) for treating diseases involving GPCR 192. In addition, the invention encompasses agonists and antagonists of GPCR 192, including small molecules, large molecules, and derivatives of the ligands that are capable of altering GPCR 192 activity. The invention also relates to methods for screening compounds (antagonists and agonists) and salts thereof that alter the binding property of ligands and the GPCR, kits for use in the screening method, compounds (antagonists and agonists) or salts thereof that alter the binding property of ligands obtainable by the screening method or obtainable using the screening kit and the GPCR protein. The invention relates to pharmaceutical compositions comprising the compounds (antagonists and agonists) that alter the binding property of ligands to the GPCR, or compounds or salts thereof that alter the expression level of the G protein coupled receptor protein.

The invention also encompasses molecules such as antisense DNA, ribozyme molecules, and antibodies to PROK1 and PROK2 that may be used to produce alterations in GPCR 192 activity. The invention also relates to methods for the use of GPCR 192, PROK1, and PROK2 for the identification of compounds and biologically active derivatives of the ligands which modulate GPCR 192 activity and are suitable for treating diseases involving GPCR 192. Such compounds may be used as therapeutic agents to treat central nervous system disorders, such as pain, metabolic disorders such as diabetes and obesity, immune disorders, and cancer. Furthermore, the invention encompasses methods of treatment and administration of the compounds identified for the treatment of diseases involving dysregulation of GPCR 192, PROK1, and PROK2.

### DESCRIPTION OF FIGURES

FIG. 1. The DNA sequence encoding the GPCR 192 polypeptide (SEQ ID NO: 1).

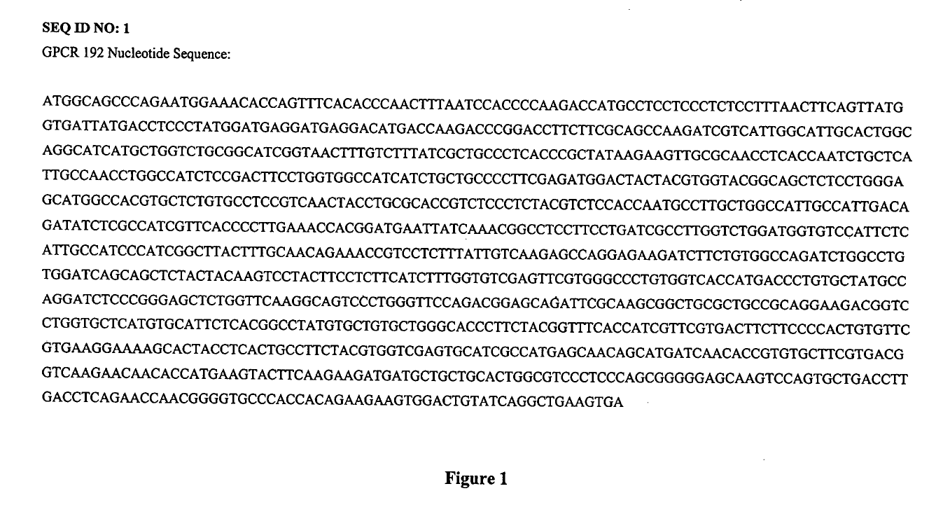

FIG. 2. The amino acid sequence of the GPCR 192 polypeptide (SEQ ID NO: 2).

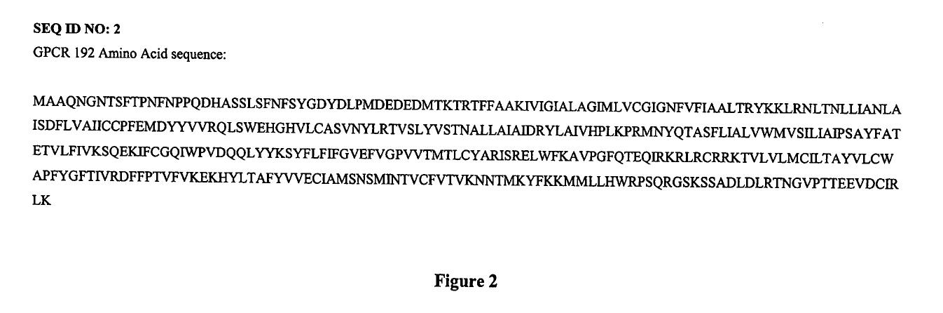

FIG. 3. The DNA sequence encoding the prokineticin 1 (PROK1) polypeptide (SEQ ID NO: 3).

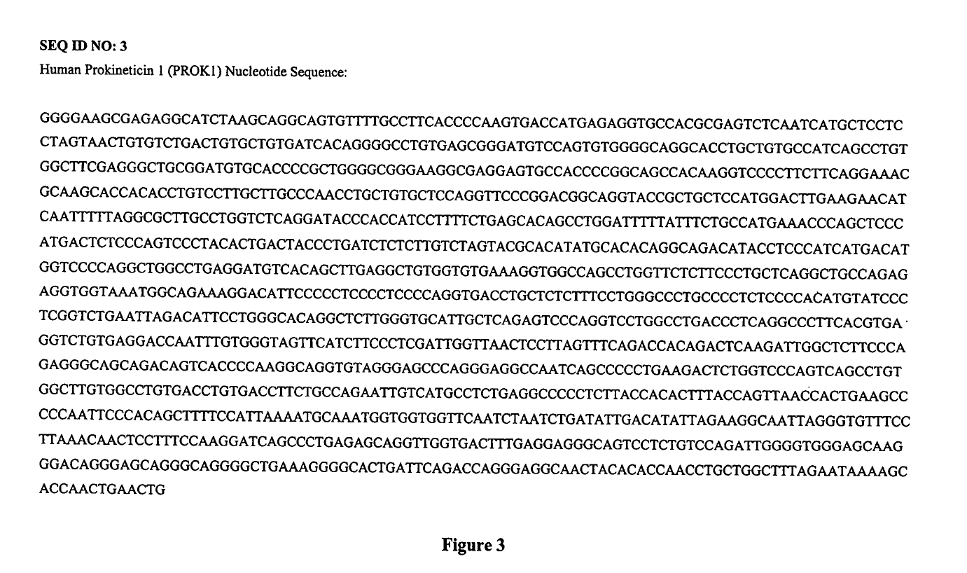

FIG. 4. The amino acid sequence of the prokineticin 1 (PROK1) polypeptide (SEQ ID NO: 4).

FIG. 5. The DNA sequence encoding the prokineticin 2 (PROK2) polypeptide (SEQ ID NO: 5).

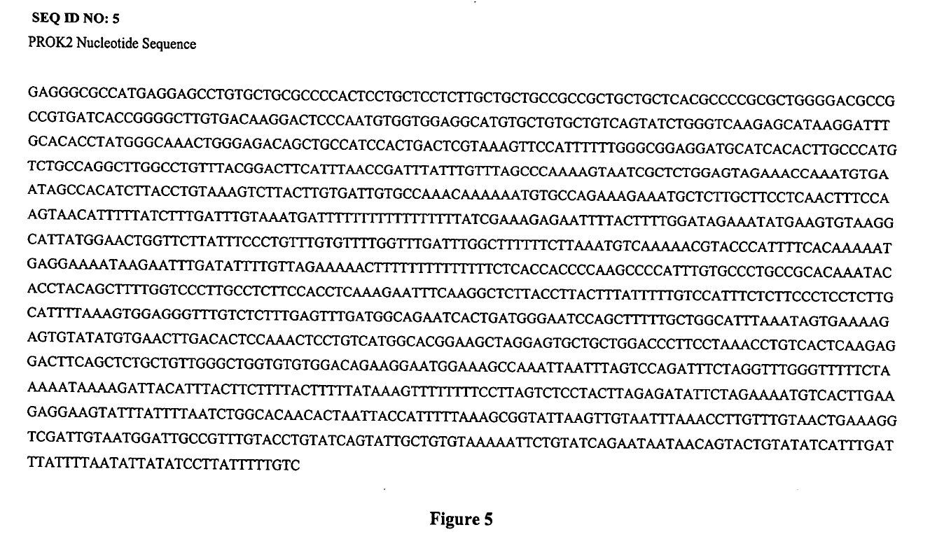

FIG. 6. The amino acid sequence of the prokineticin 2 (PROK2) polypeptide (SEQ ID NO: 6).

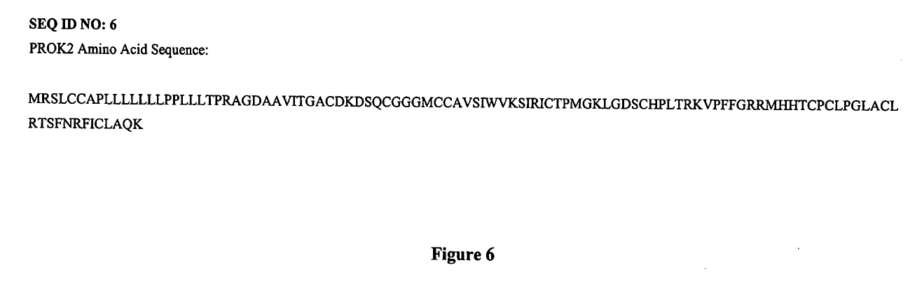

FIG. 7. The DNA sequence encoding the mouse GPR 73 polypeptide (SEQ ID NO: 7).

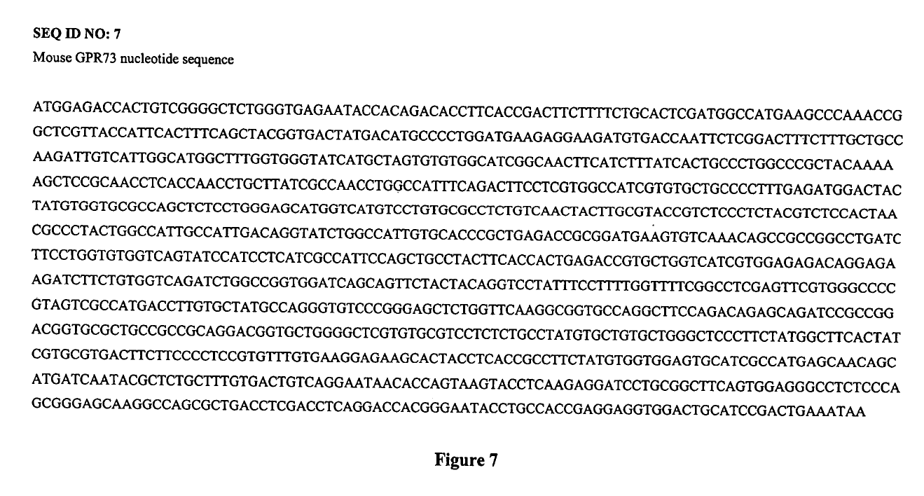

FIG. 8. The amino acid sequence of the mouse GPR 73 polypeptide (SEQ ID NO: 8).

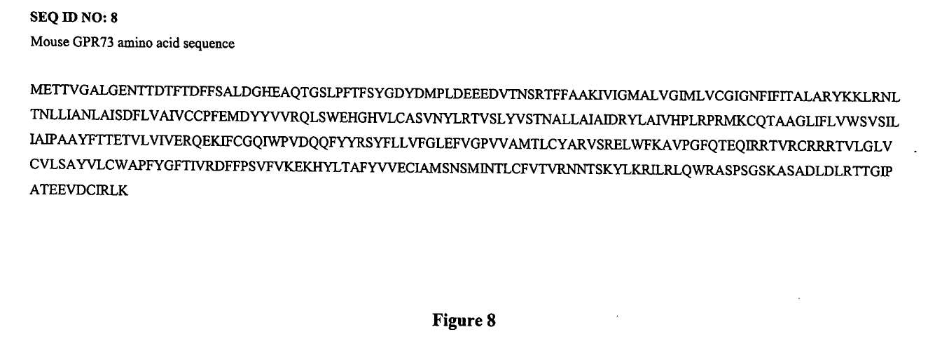

FIG. 9. The amino acid sequence of PROK1. Sequences that were identified by Edman degradation and LC/MS-MS are indicated.

FIG. 10. The activation of GPCR 192 by recombinant PROK1 and PROK2. Human embryonic kidney 293T cells were transiently transfected with empty expression vector (pcDNA3.1) or vectors encoding PROK1 or PROK2. A CHO cell-derived reporter cell line expressing GPCR 192 and a luciferase gene fused to a cAMP response element (CRE) was treated with tissue culture supernatants from the 293T transfectants and assayed for luciferase activity.

FIG. 11. Recombinant baculoviruses expressing PROK1 (rPROK1), PROK2 (rPROK2), or an unrelated, control protein were used to infect Sf9 cells. The recombinant proteins were purified and then used to treat CHO reporter cells expressing GPCR 192. Both rPROK1 and rPROK2 were capable of activating GPCR 192.

### DETAILED DESCRIPTION OF THE INVENTION

It is to be understood that this invention is not limited to the particular methodology, protocols, cell lines, animal species or genera, and reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “a cell” is a reference to one or more cells and includes equivalents thereof known to those skilled in the art, and so forth.

Unless defined otherwise, all technical and scientific terms used herein have the same meaning as commonly understood to one of ordinary skill in the art to which this invention belongs. All publications and patents mentioned herein are hereby incorporated herein by reference for the purpose of describing and disclosing, for example, the methodologies that are described in the publications which might be used in connection with the presently described invention.

The present invention relates to methods for the determination of polypeptides having ligand activity to the G protein-coupled receptor protein (GPCR) 192. Using a cell-based reporter gene assay, biological extracts were tested for the ability to activate GPCR 192. Active fractions from bovine brain extracts were identified and fractionated by standard methods yielding an apparently homogeneous preparation. The protein was identified as prokineticin 1 (PROK1), a previously described secreted protein that induces smooth muscle contraction (Li et al., Mol. Pharmacol. 59:692-698, 2001). The ability of PROK1 and a related protein, prokineticin 2 (PROK2), to active GPCR 192 was confirmed using recombinant protein from different sources.

The identification of the endogenous ligands for GPCR 192 enables the design of numerous assays for the identification of therapeutic molecules that can modulate GPCR 192 activity. For example, standard receptor binding assays may be utilized to identify compounds that affect the interaction between either PROK1 or PROK2 and GPCR 192. The source of the receptor includes membrane preparations from cells expressing the receptor or whole cells. The ligand or derivatives of the ligand (e.g., mutant versions or fragments) with similar binding properties may be labeled in a way so that specific binding to the receptor can be detected (e.g., ¹²⁵I-labeled, fluorescent tag). The binding reaction is then performed in the presence of agents that may affect the interaction between the receptor and ligand.

The identification of the endogenous ligands for GPCR 192 also enables the design of cell-based reporter gene assays for the identification of molecules that may be used to identify agents that alter the ability of the ligand to activate the receptor. The reporter gene may be an endogenous gene whose expression changes when cells expressing GPCR 192 are exposed to the ligand. Alternatively, an exogenous construct may be introduced that consists of a reporter gene (e.g., luciferase, green fluorescent protein) whose expression is regulated by a promoter sequence that is responsive to GPCR 192 signal transduction (e.g., a cyclic AMP response element CRE). The GPCR 192 reporter cells may be used to devise assays in which the activation of GPCR 192 signal transduction by either PROK1 or PROK2 induces changes in the levels of expression of the reporter gene. This type of assay can be used to identify compounds that alter the ability of PROK1 or PROK2 to interact with GPCR 192 and thus, alter its activity.

The identification of the endogenous ligands for GPCR 192 enables the design of additional cell-based assays for the identification of molecules that may be used to identify agents that alter the ability of the ligand to activate the receptor. Cell lines expressing the receptor may be used in screening assays that measure the ability of the ligand to induce changes in the intracellular levels of various second messenger molecules such as cyclic AMP (cAMP), calcium ions (Ca⁺⁺), or inositol phosphate (IP) accumulation. Such assays may be used to screen for molecules that alter the ability of the ligand to induce changes in second messenger levels.

The identification of the endogenous ligands for GPCR 192 enables the generation of various agents that may modulate the activity of GPCR 192. Altered forms of either PROK1 or PROK2 having a desirable effect on GPCR 192 activity may be identified Alternatively, fragments of either PROK1 or PROK2 (e.g., synthetic peptides) that can bind to and alter the activity of the receptor may be designed. It is also possible to generate antibodies to either PROK1, PROK2, or GPCR 192 that alter the interaction of the receptor and ligand so as to modulate GPCR 192 activity. Such agents may be used beneficial for the treatment of GPCR 192-related disorders.

Polypeptides

GPCR 192, PROK1, or PROK2 or polypeptides according to the invention comprise at least 6, 8, 10, 12, 15, 20, 25, 50, 75, 100, or more contiguous amino acids selected from the amino acid sequence shown in SEQ ID NO: 2, 4, or 6, respectively, or biologically active variants thereof, as defined below. A PROK1, PROK2, or GPCR 192 polypeptide of the invention therefore can be a portion of a PROK1, PROK2, or GPCR 192 protein, a full-length PROK1 or PROK2 protein, or a fusion protein comprising all or a portion of a PROK1, PROK2, or GPCR 192 protein.

Biologically Active Variants

PROK1, PROK2, or GPCR 192 polypeptide variants that are biologically active, e.g., retain ligand activity or receptor binding activity, also are PROK1, PROK2, or GPCR 192 polypeptides. Preferably, naturally or non-naturally occurring GPCR 192, PROK1, or PROK2 polypeptide variants have amino acid sequences which are at least about 50% identical to an amino acid sequence shown in SEQ ID NO: 2, 4, or 6, respectively, or to a fragment thereof. Percent identity between a putative GPCR 192, PROK1, or PROK2 polypeptide variant and an amino acid sequence of SEQ ID NO: 2, 4, or 6, respectively, is determined using alignment programs (e.g., Blosum62, Expect 10).

Variations in percent identity can be due, for example, to amino acid substitutions, insertions, or deletions. Amino acid substitutions are defined as one-for-one amino acid replacements. They are conservative in nature when the substituted amino acid has similar structural and/or chemical properties. Examples of conservative replacements are substitution of a leucine with an isoleucine or valine, an aspartate with a glutamate, or a threonine with a serine. Amino acid insertions or deletions are changes to or within an amino acid sequence. They typically fall in the range of about 1 to 5 amino acids. Guidance in determining which amino acid residues may be substituted, inserted, or deleted without abolishing biological or immunological activity of a PROK1, PROK2, or GPCR 192 polypeptide may be found using computer programs well known in the art, such as DNASTAR software. Whether an amino acid change results in a biologically active PROK1, PROK2, or GPCR 192 polypeptide can readily be determined by assaying for ligand activity or receptor binding activity, respectively.

Fusion Proteins

Fusion proteins are useful for generating antibodies against PROK1, PROK2, or GPCR 192 polypeptide amino acid sequences and for use in various assay systems. For example, fusion proteins can be used to identify proteins that interact with portions of a PROK1, PROK2, or GPCR 192 polypeptide. Protein affinity chromatography or library-based assays for protein-protein interactions, such as the yeast two-hybrid or phage display systems, can be used for this purpose. Such methods are well known in the art and also can be used as drug screens.

A GPCR 192, PROK1, or PROK2 polypeptide fusion protein comprises two polypeptide segments fused together by means of a peptide bond. The first polypeptide segment comprises at least 6, 8, 10, 12, 15, 20, 25, 50, 75, 100, or more contiguous amino acids of SEQ ID NO: 2, 4, or 6, respectively, or of a biologically active variant, such as those described above. The first polypeptide segment also can comprise full-length PROK1, PROK2, or GPCR 192 protein.

The second polypeptide segment can be a full-length protein or a protein fragment. Proteins commonly used in fusion protein construction include β-galactosidase, glucuronidase, green fluorescent protein (GFP), autofluorescent proteins, including blue fluorescent protein (BFP), glutathione-S-transferase (GST), luciferase, horseradish peroxidase (HRP), and chloramphenicol acetyltransferase (CAT). Additionally, epitope tags may be used in fusion protein constructions, including histidine (His tags), FLAG tags, influenza hemagglutinin (HA) tags, Myc tags, VSV-G tags, and thioredoxin (Trx) tags. Other fusion constructions may include maltose binding protein (MBP), S-tag, Lex A DNA binding domain (DBD) fusions, GAL4 DNA binding domain fusions, and herpes simplex virus (HSV) BP16 protein fusions. A fusion protein also may be engineered to contain a cleavage site located between the PROK1, PROK2, or GPCR 192 polypeptide-encoding sequence and the heterologous protein sequence, so that the PROK1, PROK2, or GPCR 192 polypeptide can be cleaved and purified away from the heterologous moiety.

A fusion protein may be synthesized chemically, as is known in the art. Preferably, a fusion protein is produced by covalently linking two polypeptide segments or by standard procedures in the art of molecular biology. Recombinant DNA methods can be used to prepare fusion proteins, for example, by making a DNA construct which comprises coding sequences selected from SEQ ID NO: 1, 3, or 5 in proper reading frame with nucleotides encoding the second polypeptide segment and expressing the DNA construct in a host cell, as is known in the art. Many kits for constructing fusion proteins are available from companies such as Promega Corporation (Madison, Wis.), Stratagene (La Jolla, Calif.), CLONTECH (Mountain View, Calif., Santa Cruz Biotechnology (Santa Cruz, Calif.), MBL International Corporation (MIC; Watertown, Mass.), and Quantum Biotechnologies (Montreal, Canada).

Identification of Species Homologs

Species homologs of the PROK1, PROK2, or GPCR 192 polypeptides disclosed herein may be obtained using PROK1, PROK2, or GPCR 192 polynucleotides to make suitable probes or primers for screening cDNA expression libraries from other species, such as mice, monkeys, or yeast, identifying cDNAs which encode homologs of the PROK1, PROK2, or GPCR 192 polypeptide, and expressing the cDNAs as is known in the art.

Polynucleotides.

A PROK1, PROK2, or GPCR 192 polynucleotide may be single- or double-stranded and comprises a coding sequence or the complement of a coding sequence for a PROK1, PROK2, or GPCR 192 polypeptide. The coding sequence for GPCR 192 is shown in SEQ ID NO: 1; the coding sequence for PROK1 is shown in SEQ ID NO: 3; and the coding sequence for PROK2 is shown in SEQ ID NO: 5. Degenerate nucleotide sequences encoding GPCR 192, PROK1, or PROK2 polypeptides, as well as homologous nucleotide sequences which are at least about 50, 55, 60, 65, 70, preferably about 75, 90, 96, 98, or 99% identical to the nucleotide sequences shown in SEQ ID NO: 1, 3, or 5, respectively, or their complements also are GPCR 192, PROK1, or PROK2 polynucleotides. Percent sequence identity between the sequences of two polynucleotides is determined using computer programs such as ALIGN which employ the FASTA algorithm, using an affine gap search with a gap open penalty of −12 and a gap extension penalty of −2. Complementary DNA (cDNA) molecules, species homologs, and variants of PROK1, PROK2, or GPCR 192 polynucleotides that encode biologically active PROK1, PROK2, or GPCR 192 polypeptides also are PROK1, PROK2, or GPCR 192 polynucleotides. Polynucleotide fragments comprising at least 8, 9, 10, 11, 12, 15, 20, or 25 contiguous nucleotides of SEQ ID NO: 1, 3, or 5, or its complements also are GPCR 192, PROK1, or PROK2 polynucleotides, respectively. These fragments can be used, for example, as hybridization probes or as antisense oligonucleotides.

Identification of Polynucleotide Variants and Homologs

Variants and homologs of the PROK1, PROK2, or GPCR 192 polynucleotides described above also are PROK1, PROK2, or GPCR 192 polynucleotides. Typically, homologous PROK1, PROK2, or GPCR 192 polynucleotide sequences may be identified by hybridization of candidate polynucleotides to PROK1, PROK2, or GPCR 192 polynucleotides under stringent conditions, as is known in the art. For example, using the following wash conditions: 2×SSC (0.3 M NaCl, 0.03 M sodium citrate, pH 7.0), 0.1% SDS, room temperature twice, 30 minutes each; then 2×SSC, 0.1% SDS, 50° C. once, 30 minutes; then 2×SSC, room temperature twice, 10 minutes each; homologous sequences can be identified which contain at most about 25-30% basepair mismatches. More preferably, homologous nucleic acid strands contain 15-25% basepair mismatches, even more preferably 5-15% basepair mismatches.

Species homologs of the PROK1, PROK2, or GPCR 192 polynucleotides disclosed herein may also be identified by making suitable probes or primers and screening cDNA expression libraries from other species, such as mice, monkeys, or yeast. Human variants of PROK1, PROK2, or GPCR 192 polynucleotides can be identified, for example, by screening human cDNA expression libraries.

It is well known that the Tm of a double-stranded DNA decreases by 1-1.5° C. with every 1% decrease in homology (Boiner et al., J. Mol. Biol. 81:123, 1973). Variants of human GPCR 192, PROK1, or PROK2 polynucleotides or GPCR 192, PROK1, or PROK2 polynucleotides of other species can therefore be identified by hybridizing a putative homologous GPCR 192, PROK1, or PROK2 polynucleotide with a polynucleotide having a nucleotide sequence of SEQ ID NO: 1, 3, or 5, respectively, or the complement thereof to form a test hybrid. The melting temperature of the test hybrid is compared with the melting temperature of a hybrid comprising polynucleotides having perfectly complementary nucleotide sequences, and the number or percent of basepair mismatches within the test hybrid is calculated.

Nucleotide sequences which hybridize to PROK1, PROK2, or GPCR 192 polynucleotides or their complements following stringent hybridization and/or wash conditions also are PROK1, PROK2, or GPCR 192 polynucleotides. Stringent wash conditions are well known and understood in the art and are disclosed, for example, in Sambrook et al., (MOLECULAR CLONING: A LABORATORY MANUAL, 2d ed., 1989, at pages 9.50-9.51).

Typically, for stringent hybridization conditions, a combination of temperature and salt concentration should be chosen that is approximately 12-20° C. below the calculated Tm of the hybrid under study. The Tm of a hybrid between a GPCR 192, PROK1, or PROK2 polynucleotide having a nucleotide sequence shown in SEQ ID NO: 1, 3, or 5, respectively, or the complement thereof and a polynucleotide sequence which is at least about 50, preferably about 75, 90, 96, or 98% identical to one of those nucleotide sequences can be calculated, for example, using the equation of Bolton and McCarthy (Proc. Natl. Acad. Sci. U.S.A. 48:1390, 1962):
Tₘ=81.5° C.−16.6(log₁₀[Na+])+0.41(% G+C)−0.63(% formamide)−600/l),
where l=the length of the hybrid in basepairs.

Stringent wash conditions include, for example, 4×SSC at 65° C., or 50% formamide, 4×SSC at 42° C., or 0.5×SSC, 0.1% SDS at 65° C. Highly stringent wash conditions include, for example, 0.2×SSC at 65° C.

Preparation of Polynucleotides.

A PROK1, PROK2, or GPCR 192 polynucleotide may be isolated free of other cellular components such as membrane components, proteins, and lipids. Polynucleotides may be made by a cell and isolated using standard nucleic acid purification techniques, or synthesized using an amplification technique, such as the polymerase chain reaction (PCR), or by using an automatic synthesizer. Methods for isolating polynucleotides are routine and are known in the art. Any such technique for obtaining a polynucleotide can be used to obtain isolated PROK1, PROK2, or GPCR 192 polynucleotides. For example, restriction enzymes and probes can be used to isolate polynucleotide fragments, which comprise PROK1, PROK2, or GPCR 192 nucleotide sequences. Isolated polynucleotides are in preparations that are free or at least 70, 80, or 90% free of other molecules. PROK1, PROK2, or GPCR 192 cDNA molecules can be made with standard molecular biology techniques, using PROK1, PROK2, or GPCR 192 mRNA as a template. cDNA molecules can thereafter be replicated using molecular biology techniques known in the art and disclosed in manuals such as Sambrook et al., (1989). An amplification technique, such as PCR, can be used to obtain additional copies of polynucleotides of the invention, using either human genomic DNA or cDNA as a template.

Alternatively, synthetic chemistry techniques can be used to synthesize PROK1, PROK2, or GPCR 192 polynucleotides. The degeneracy of the genetic code allows alternate nucleotide sequences to be synthesized which will encode a GPCR 192, PROK1, or PROK2 polypeptide having, for example, an amino acid sequence shown in SEQ ID NO: 2, 4, or 6, respectively, or a biologically active variant thereof.

Extension of Polynucleotides

Various PCR-based methods can be used to extend the nucleic acid sequences disclosed herein to detect upstream sequences such as promoters and regulatory elements. For example, restriction-site PCR uses universal primers to retrieve unknown sequence adjacent to a known locus (Sarkar, PCR Methods Applic. 2:318-322, 1993). Genomic DNA is first amplified in the presence of a primer to a linker sequence and a primer specific to the known region. The amplified sequences are then subjected to a second round of PCR with the same linker primer and another specific primer internal to the first one. Products of each round of PCR are transcribed with an appropriate RNA polymerase and sequenced using reverse transcriptase.

Inverse PCR also can be used to amplify or extend sequences using divergent primers based on a known region (Triglia et al., Nucleic Acids Res. 16:8186, 1988). Primers can be designed using commercially available software, such as OLIGO 4.06 Primer Analysis software (National Biosciences Inc., Plymouth, Minn.), to be 22-30 nucleotides in length, to have a GC content of 50% or more, and to anneal to the target sequence at temperatures about 65-72° C. The method uses several restriction enzymes to generate a suitable fragment in the known region of a gene. The fragment is then circularized by intramolecular ligation and used as a PCR template.

Another method which can be used is capture PCR, which involves PCR amplification of DNA fragments adjacent to a known sequence in human and yeast artificial chromosome DNA (Lagerstrom et al., PCR Methods Applic. 1:111-119, 1991). In this method, multiple restriction enzyme digestions and ligations also can be used to place an engineered double-stranded sequence into an unknown fragment of the DNA molecule before performing PCR. Another method which may be used to retrieve unknown sequences is that of Parker et al., (Nucleic Acids Res. 19:3055-3060, 1991). Additionally, PCR, nested primers, and PROMOTERFINDER libraries (CLONTECH, Palo Alto, Calif.) can be used to walk genomic DNA (CLONTECH, Palo Alto, Calif.). This process avoids the need to screen libraries and is useful in finding intron/exon junctions.

When screening for full-length cDNAs, it is preferable to use libraries that have been size-selected to include larger cDNAs. Randomly-primed libraries are preferable, in that they will contain more sequences which contain the 5′ regions of genes. Use of a randomly primed library may be especially preferable for situations in which an oligo d(T) library does not yield a full-length cDNA. Genomic libraries can be useful for extension of sequence into 5′ non-transcribed regulatory regions.

Commercially available capillary electrophoresis systems can be used to analyze the size or confirm the nucleotide sequence of PCR or sequencing products. For example, capillary sequencing can employ flowable polymers for electrophoretic separation, four different fluorescent dyes (one for each nucleotide) that are laser activated, and detection of the emitted wavelengths by a charge coupled device camera. Output/light intensity can be converted to electrical signal using appropriate software (e.g., GENOTYPER and Sequence NAVIGATOR, Perkin Elmer), and the entire process from loading of samples to computer analysis and electronic data display can be computer controlled. Capillary electrophoresis is especially preferable for the sequencing of small pieces of DNA that might be present in limited amounts in a particular sample.

Obtaining Polypeptides

PROK1, PROK2, or GPCR 192 polypeptides can be obtained, for example, by purification from mammalian cells, by expression of PROK1, PROK2, or GPCR 192 polynucleotides, or by direct chemical synthesis.

Protein Purification.

PROK1, PROK2, or GPCR 192 polypeptides can be purified from any cell that expresses the polypeptide, including host cells that have been transfected with PROK1, PROK2, or GPCR 192 expression constructs. A purified PROK1, PROK2, or GPCR 192 polypeptide is separated to from other compounds that normally associate with the PROK1, PROK2, or GPCR 192 polypeptide in the cell, such as certain proteins, carbohydrates, or lipids, using methods well-known in the art. Such methods include, but are not limited to, size exclusion chromatography, ammonium sulfate fractionation, ion exchange chromatography, affinity chromatography, and preparative gel electrophoresis. A preparation of purified PROK1, PROK2, or GPCR 192 polypeptides is at least 80% pure; preferably, the preparations are 90%, 95%, or 99% pure. Purity of the preparations can be assessed by any means known in the art, such as SDS-polyacrylamide gel electrophoresis.

Expression of Polynucleotides

To express a PROK1, PROK2, or GPCR 192 polynucleotide, the polynucleotide may be inserted into an expression vector that contains the necessary elements for the transcription and translation of the inserted coding sequence. Methods that are well known to those skilled in the art can be used to construct expression vectors containing sequences encoding PROK1, PROK2, or GPCR 192 polypeptides and appropriate transcriptional and translational control elements. These methods include in vitro recombinant DNA techniques, synthetic techniques, and in vivo genetic recombination. Such techniques are described, for example, in Sambrook et al. (1989) and in Ausubel et al., CURRENT PROTOCOLS MOLECULAR BIOLOGY, John Wiley & Sons, New York, N.Y., 1989.

A variety of expression vector/host systems can be utilized to contain and express sequences encoding a PROK1, PROK2, or GPCR 192 polypeptide. These include, but are not limited to, microorganisms, such as bacteria transformed with recombinant bacteriophage, plasmid, or cosmic DNA expression vectors; yeast transformed with yeast expression vectors; insect cell systems infected with virus expression vectors (e.g., baculovirus); plant cell systems transformed with virus expression vectors (e.g., cauliflower mosaic virus, CaMV; tobacco mosaic virus, TMV); or with bacterial expression vectors (e.g., Ti or pBR322 plasmids); or animal cell systems.

The control elements or regulatory sequences are those non-translated regions of the vector—enhancers, promoters, 5′ and 3′ untranslated regions—which interact with host cellular proteins to carry out transcription and translation. Such elements can vary in their strength and specificity. Depending on the vector system and host utilized, any number of suitable transcription and translation elements, including constitutive and inducible promoters, can be used. For example, when cloning in bacterial systems, inducible promoters such as the hybrid lacZ promoter of the BLUESCRIPT phagemid (Stratagene, LaJolla, Calif.) or pSPORT1 plasmid (Life Technologies) and the like can be used. The baculovirus polyhedrin promoter can be used in insect cells. Promoters or enhancers derived from the genomes of plant cells (e.g., heat shock, RUBISCO, and storage protein genes) or from plant viruses (e.g., viral promoters or leader sequences) can be cloned into the vector. In mammalian cell systems, promoters from mammalian genes or from mammalian viruses are preferable. If it is necessary to generate a cell line that contains multiple copies of a nucleotide sequence encoding a PROK1, PROK2, or GPCR 192 polypeptide, vectors based on SV40 or EBV can be used with an appropriate selectable marker.

Bacterial and Yeast Expression Systems.

In bacterial systems, a number of expression vectors can be selected depending upon the use intended for the PROK1, PROK2, or GPCR 192 polypeptide. For example, when a large quantity of a PROK1, PROK2, or GPCR 192 polypeptide is needed for the induction of antibodies, vectors which direct high level expression of fusion proteins that are readily purified can be used. Such vectors include, but are not limited to, multifunctional E. coli cloning and expression vectors such as BLUESCRIPT (Stratagene). In a BLUESCRIPT vector, a sequence encoding the PROK1 or PROK2 polypeptide can be ligated into the vector in frame with sequences for the amino-terminal Met and the subsequent residues of β-galactosidase so that a hybrid protein is produced. pIN vectors (Van Heels and Schuster, J. Biol. Chem. 264:5503-5509, 1989) or pGEX vectors (Promega, Madison, Wis.) also can be used to express foreign polypeptides as fusion proteins with glutathione S-transferase (GST). In general, such fusion proteins are soluble and can easily be purified from lysed cells by adsorption to glutathione-agarose beads followed by elusion in the presence of free glutathione. Proteins made in such systems can be designed to include heparin, thrombin, or factor Xa protease cleavage sites so that the cloned polypeptide of interest can be released from the GST moiety at will.

In the yeast Saccharomyces cerevisiae, a number of vectors containing constitutive or inducible promoters such as alpha factor, alcohol oxidase, and PGH can be used. For reviews, see, for example, Ausubel et al., (1989) and Grant et al., (Methods Enzymol. 153:516-544, 1987).

Plant and Insect Expression Systems.

If plant expression vectors are used, the expression of sequences encoding PROK1, PROK2, or GPCR 192 polypeptides can be driven by any of a number of promoters. For example, viral promoters such as the 35S and 19S promoters of CaMV can be used alone or in combination with the omega leader sequence from TMV (Takamatsu, EMBO J. 6:307-311, 1987). Alternatively, plant promoters such as the small subunit of RUBISCO or heat shock promoters can be used (Coruzzi et al., EMBO J. 3: 1671-16SO, 1984, Broglie et al., Science 924:833-843, 1984; Winter et al., Results Probl. Cell Differ. 17:S5-105, 1991). These constructs can be introduced into plant cells by direct DNA transformation or by pathogen-mediated transfection. Such techniques are described in a number of generally available reviews (e.g., Hobbs or Murray, in MCGRAW HILL YEARBOOK OF SCIENCE AND TECHNOLOGY, McGraw Hill, New York, N.Y., pp. 191-196, 1992).

An insect system also can be used to express a PROK1, PROK2, or GPCR 192 polypeptide. For example, in one such system Autographa californica nuclear polyhedrosis virus (AcNPV) is used as a vector to express foreign genes in Spodoptera frugiperda cells or in Trichoplusia larvae. Sequences encoding PROK1, PROK2, or GPCR 192 polypeptides can be cloned into a non-essential region of the virus, such as the polyhedrin gene, and placed under control of the polyhedrin promoter. Successful insertion of PROK1, PROK2, or GPCR 192 polypeptides will render the polyhedrin gene inactive and produce recombinant virus lacking coat protein. The recombinant viruses can then be used to infect S. frugiperda cells or Trichoplusia larvae in which PROK1, PROK2, or GPCR 192 polypeptides can be expressed (Engelhard et al., Proc. Nat. Acad. Sci. 91:3224-3227, 1994).

Mammalian Expression Systems.

A number of viral-based expression systems can be used to express PROK1, PROK2, or GPCR 192 polypeptides in mammalian host cells. For example, if an adenovirus is used as an expression vector, sequences encoding PROK1, PROK2, or GPCR 192 polypeptides can be ligated into an adenovirus transcription/translation complex comprising the late promoter and tripartite leader sequence.

Insertion in a non-essential E1 or E3 region of the viral genome can be used to obtain a viable virus that is capable of expressing a PROK1, PROK2, or GPCR 192 polypeptide in infected host cells (Logan and Shenk, Proc. Natl. Acad. Sci. 81:3655-3659, 1984). If desired, transcription enhancers, such as the Rous sarcoma virus (RSV) enhancer, can be used to increase expression in mammalian host cells. Human artificial chromosomes (HACs) also can be used to deliver larger fragments of DNA than can be contained and expressed in a plasmid. HACs of 6M to 10M are constructed and delivered to cells via conventional delivery methods (e.g., liposomes, polycationic amino polymers, or vesicles).

Specific initiation signals also can be used to achieve more efficient translation of sequences encoding PROK1, PROK2, or GPCR 192 polypeptides. Such signals include the ATG initiation codon and adjacent sequences. In cases where sequences encoding a PROK1, PROK2, or GPCR 192 polypeptide, its initiation codon, and upstream sequences are inserted into the appropriate expression vector, no additional transcriptional or translational control signals may be needed. However, in cases where only coding sequence, or a fragment thereof, is inserted, exogenous translational control signals (including the ATG initiation codon) should be provided. The initiation codon should be in the correct reading frame to ensure translation of the entire insert. Exogenous translational elements and initiation codons can be of various origins, both natural and synthetic. The efficiency of expression can be enhanced by the inclusion of enhancers which are appropriate for the particular cell system which is used (see, e.g., Scharf et al., Results Probl. Cell Differ. 20:125-162, 1994).

Host Cells.

A host cell strain can be chosen for its ability to modulate the expression of the inserted sequences or to process the expressed PROK1, PROK2, or GPCR 192 polypeptide in the desired fashion. Such modifications of the polypeptide include, but are not limited to, acetylation, carboxylation, glycosylation, phosphorylation, lipidation, and acylation. Post-translational processing which cleaves a “prepro” form of the polypeptide also can be used to facilitate correct insertion, folding, and/or function. Different host cells that have specific cellular machinery and characteristic mechanisms for post-translational activities (e.g., CHO, HeLa, MDCK, HEK293, and WI3S), are available from the American Type Culture Collection (ATCC, Manassas, Va.) and can be chosen to ensure the correct modification and processing of the foreign protein.

Stable expression is preferred for long-term, high-yield production of recombinant proteins. For example, cell lines which stably express PROK1, PROK2, or GPCR 192 polypeptides can be transformed using expression vectors which can contain viral origins of replication and/or endogenous expression elements and a selectable marker gene on the same or on a separate vector. Following the introduction of the vector, cells can be allowed to grow for 1-2 days in an enriched medium before they are switched to a selective medium. The purpose of the selectable marker is to confer resistance to selection, and its presence allows growth and recovery of cells which successfully express the introduced PROK1, PROK2, or GPCR 192 sequences. Resistant clones of stably transformed cells can be proliferated using tissue culture techniques appropriate to the cell type. See, for example, ANIMAL CELL CULTURE, R. I. Freshney, ed., 1986.

Any number of selection systems can be used to recover transformed cell lines. These include, but are not limited to, the herpes simplex virus thymidine kinase (Wigler et al., Cell 11:223-32, 1977) and adenine phosphoribosyltransferase (Lowy et al., Cell 22:817-23, 1980) genes that can be employed in tk⁻ or aprt⁻ cells, respectively. Also, antimetabolite, antibiotic, or herbicide resistance can be used as the basis for selection. For example, dhfr confers resistance to methotrexate (Wigler et al., Proc. Natl. Acad. Sci. 77:3567-70, 1980), npt confers resistance to the aminoglycosides, neomycin, and G418 (Colbere-Garapin et al., J. Mol. Biol. 150:1-14, 1981), and als and pat confer resistance to chlorsulfuron and phosphinotricin acetyltransferase, respectively (Murray, 1992). Additional selectable genes have been described. For example, trpB allows cells to utilize indole in place of tryptophan, or hisD, which allows cells to utilize histinol in place of histidine (Hartman and Mulligan, Proc. Natl. Acad. Sci. 85:8047-51, 1988). Visible markers such as anthocyanins, β-glucuronidase and its substrate GUS, and luciferase and its substrate luciferin, can be used to identify transformants and to quantify the amount of transient or stable protein expression attributable to a specific vector system (Rhodes et al., Methods Mol. Biol. 55:121-131, 1995).

Detecting Expression

Although the presence of marker gene expression suggests that the PROK1, PROK2, or GPCR 192 polynucleotide is also present, its presence and expression may need to be confirmed. For example, if a sequence encoding a PROK1, PROK2, or GPCR 192 polypeptide is inserted within a marker gene sequence, transformed cells containing sequences that encode a PROK1, PROK2, or GPCR 192 polypeptide can be identified by the absence of marker gene function.

Alternatively, a marker gene can be placed in tandem with a sequence encoding a PROK1, PROK2, or GPCR 192 polypeptide under the control of a single promoter. Expression of the marker gene in response to induction or selection usually indicates expression of the PROK1, PROK2, or GPCR 192 polynucleotide.

Alternatively, host cells which contain a PROK1, PROK2, or GPCR 192 polynucleotide and which express a PROK1, PROK2, or GPCR 192 polypeptide can be identified by a variety of procedures known to those of skill in the art. These procedures include, but are not limited to, DNA-DNA or DNA-RNA hybridizations and protein bioassay or immunoassay techniques that include membrane, solution, or chip-based technologies for the detection and/or quantification of nucleic acid or protein. For example, the presence of a polynucleotide sequence encoding a PROK1, PROK2, or GPCR 192 polypeptide can be detected by DNA-DNA or DNA-RNA hybridization or amplification using probes or fragments or fragments of polynucleotides encoding a PROK1, PROK2, or GPCR 192 polypeptide.

Nucleic acid amplification-based assays involve the use of oligonucleotides selected from sequences encoding a PROK1, PROK2, or GPCR 192 polypeptide to detect transformants that contain a PROK1, PROK2, or GPCR 192 polynucleotide.

A variety of protocols for detecting and measuring the expression of a PROK1, PROK2, or GPCR 192 polypeptide, using either polyclonal or monoclonal antibodies specific for the polypeptide, are known in the art. Examples include enzyme-linked immunosorbent assay (ELISA), radioimmunoassay (RIA), and fluorescence activated cell sorting (FACS). A two-site, monoclonal-based immunoassay using monoclonal antibodies reactive to two non-interfering epitopes on a PROK1, PROK2, or GPCR 192 polypeptide can be used, or a competitive binding assay can be employed. These and other assays are described in Hampton et al., (SEROLOGICAL METHODS: A LABORATORY MANUAL, APS Press, St. Paul, Minn., 1990) and Maddox et al., (J. Exp. Med. 158:1911-1216, 1983).

A wide variety of labels and conjugation techniques are known by those skilled in the art and can be used in various nucleic acid and amino acid assays. Means for producing labeled hybridization or PCR probes for detecting sequences related to polynucleotides encoding PROK1, PROK2, or GPCR 192 polypeptides include oligolabeling, nick translation, end-labeling, or PCR amplification using a labeled nucleotide. Alternatively, sequences encoding a PROK1, PROK2, or GPCR 192 polypeptide can be cloned into a vector for the production of an mRNA probe.

Such vectors are known in the art, are commercially available, and can be used to synthesize RNA probes by addition of labeled nucleotides and an appropriate RNA polymerase such as T7, T3, or SP6. These procedures can be conducted using a variety of commercially available kits (Amersham Pharmacia Biotech, Promega, and US Biochemical). Suitable reporter molecules or labels which can be used for ease of detection include radionuclides, enzymes, and fluorescent, chemiluminescent, or chromogenic agents, as well as substrates, cofactors, inhibitors, magnetic particles, and the like.

Expression and Purification of Polypeptides

Host cells transformed with nucleotide sequences encoding a PROK1, PROK2, or GPCR 192 polypeptide can be cultured under conditions suitable for the expression and recovery of the protein from cell culture. The polypeptide produced by a transformed cell can be secreted or contained intracellularly depending on the sequence and/or the vector used. As will be understood by those of skill in the art, expression vectors containing polynucleotides which encode PROK1, PROK2, or GPCR 192 polypeptides can be designed to contain signal sequences which direct secretion of soluble PROK1, PROK2, or GPCR 192 polypeptides through a prokaryotic or eukaryotic cell membrane or which direct the membrane insertion of PROK1, PROK2, or GPCR 192 polypeptide.

As discussed above, other constructions can be used to join a sequence encoding a PROK1, PROK2, or GPCR 192 polypeptide to a nucleotide sequence encoding a polypeptide domain which will facilitate purification of soluble proteins. Such purification facilitating domains include, but are not limited to, metal chelating peptides such as histidine-tryptophan modules that allow purification on immobilized metals, protein A domains that allow purification on immobilized immunoglobulin, and the domain utilized in the FLAGS extension/affinity purification system (Immunex Corp., Seattle, Wash.). Inclusion of cleavable linker sequences such as those specific for Factor Xa or enterokinase (Invitrogen, San Diego, Calif.) between the purification domain and the PROK1, PROK2, or GPCR 192 polypeptide also can be used to facilitate purification. One such expression vector provides for expression of a fusion protein containing a PROK1, PROK2, or GPCR 192 polypeptide and six histidine residues preceding a thioredoxin or an enterokinase cleavage site.

The histidine residues facilitate purification by IMAC (immobilized metal ion affinity chromatography, as described in Porath et al., (Prot. Exp. Purif. 3:263-81, 1992) while the enterokinase cleavage site provides a means for purifying the PROK1, PROK2, or GPCR 192 polypeptide from the fusion protein. Vectors that contain fusion proteins are disclosed in Kroll et al., (DNA Cell. Biol. 19:441453, 1993).

Chemical Synthesis

Sequences encoding a PROK1, PROK2, or GPCR 192 polypeptide can be synthesized, in whole or in part, using chemical methods well known in the art (see, e.g., Caruthers et al., Nucl. Acids Res. Symp. Ser. 215-223, 1980; Horn et al., Nucl. Acids Res. Symp Ser 215-223, 1980). Alternatively, a PROK1, PROK2, or GPCR 192 polypeptide itself can be produced using chemical methods to synthesize its amino acid sequence, such as by direct peptide synthesis using solid-phase techniques (Merrifield, J. Am. Chem. Soc. 85:2149-2154, 1963; Roberge et al., Science 269:202-204, 1995). Protein synthesis can be performed using manual techniques or by automation. Automated synthesis can be achieved, for example, using Applied Biosystems 431A Peptide Synthesizer (Perkin Elmer). Optionally, fragments of PROK1, PROK2, or GPCR 192 polypeptides can be separately synthesized and combined using chemical methods to produce a full-length molecule.

The newly synthesized peptide can be substantially purified by preparative high performance liquid chromatography (e.g., Creighton, PROTEINS: STRUCTURES AND MOLECULAR PRINCIPLES, WH Freeman and Co., New York, N.Y., 1983). The composition of a synthetic PROK1, PROK2, or GPCR 192 polypeptide can be confirmed by amino acid analysis or sequencing (e.g., the Edman degradation procedure; see Creighton, supra). Additionally, any portion of the amino acid sequence of the PROK1, PROK2, or GPCR 192 polypeptide can be altered during direct synthesis and/or combined using chemical methods with sequences from other proteins to produce a variant polypeptide or a fusion protein.

Production of Altered Polypeptides

As will be understood by those of skill in the art, it may be advantageous to produce PROK1, PROK2, or GPCR 192 polypeptide-encoding nucleotide sequences possessing non-naturally occurring codons. For example, codons preferred by a particular prokaryotic or eukaryotic host can be selected to increase the rate of protein expression or to produce an RNA transcript having desirable properties, such as a half-life that is longer than that of a transcript generated from the naturally occurring sequence.

The nucleotide sequences disclosed herein can be engineered using methods generally known in the art to alter PROK1, PROK2, or GPCR 192 polypeptide-encoding sequences for a variety of reasons, including but not limited to, alterations which modify the cloning, processing, and/or expression of the polypeptide or mRNA product. DNA shuffling by random fragmentation and PCR reassembly of gene fragments and synthetic oligonucleotides can be used to engineer the nucleotide sequences. For example, site-directed mutagenesis can be used to insert new restriction sites, alter glycosylation patterns, change codon preference, produce splice variants, introduce mutations, and so forth.

Antibodies

Any type of antibody known in the art can be generated to bind specifically to an epitope of a PROK1, PROK2, or GPCR 192 polypeptide. “Antibody” as used herein includes intact immunoglobulin molecules, as well as fragments thereof, such as Fab, F(ab′)2, and Fv, which are capable of binding an epitope of a PROK1, PROK2, or GPCR 192 polypeptide. Typically, at least 6, 8, 10, or 12 contiguous amino acids are required to form an epitope. However, epitopes which involve non-contiguous amino acids may require more, for example, at least 15, 25, or 50 amino acids.

An antibody which specifically binds to an epitope of a PROK1, PROK2, or GPCR 192 polypeptide can be used therapeutically, as well as in immunochemical assays, such as Western blots, ELISAs, radioimmunoassays, immunohistochemical assays, immunoprecipitations, or other immunochemical assays known in the art. Various immunoassays can be used to identify antibodies having the desired specificity. Numerous protocols for competitive binding or immunoradiometric assays are well known in the art. Such immunoassays typically involve the measurement of complex formation between an immunogen and an antibody that specifically binds to the immunogen.

Typically, an antibody which specifically binds to a PROK1, PROK2, or GPCR 192 polypeptide provides a detection signal at least 5-, 10-, or 20-fold higher than a detection signal provided with other proteins when used in an immunochemical assay. Preferably, antibodies which specifically bind to PROK1, PROK2, or GPCR 192 polypeptides do not detect other proteins in immunochemical assays and can immunoprecipitate a PROK1, PROK2, or GPCR 192 polypeptide from solution. Most preferably, the antibodies are neutralizing antibodies, which inhibit the activity of PROK1, PROK2, or GPCR 192.

Human PROK1, PROK2, or GPCR 192 polypeptides can be used to immunize a mammal, such as a mouse, rat, rabbit, guinea pig, monkey, or human, to produce polyclonal antibodies. If desired, a PROK1, PROK2, or GPCR 192 polypeptide can be conjugated to a carrier protein, such as bovine serum albumin, thyroglobulin, and keyhole limpet hemocyanin. Depending on the host species, various adjuvants can be used to increase the immunological response. Such adjuvants include, but are not limited to, Freund's adjuvant, mineral gels (e.g., aluminum hydroxide), and surface active substances (e.g., lysolecithin, pluronic polyols, polyanions, peptides, oil emulsions, keyhole limpet hemocyanin, and dinitrophenol). Among adjuvants used in humans, BCG 25 (bacilli Calmette-Guerin) and Cornyebacterium parvum are especially useful.

Monoclonal antibodies that specifically bind to a PROK1, PROK2, or GPCR 192 polypeptide can be prepared using any technique which provides for the production of antibody molecules by continuous cell lines in culture. These techniques include, but are not limited to, the hybridoma technique, the human B-cell hybridoma technique, and the EBV-hybridoma technique (Kohler et al., Nature 56:495-497, 1985; Kozbor et al., J. Immunol. Methods 81:31-42, 1985; Cote et al., Proc. Natl. Acad. Sci. 80:2026-2030, 1993; Cole et al., Mol. Cell Biol. 62:109-120, 1984).

In addition, techniques developed for the production of “chimeric antibodies,” the splicing of mouse antibody genes to human antibody genes to obtain a molecule with appropriate antigen specificity and biological activity, can be used (Morrison et al., Proc. Natl. Acad. Sci. 81:6851-6855, 1984; Neuberger et al., Nature 312:604-608, 1984; Takeda et al., Nature 314:452-454, 1985). Monoclonal and other antibodies also can be “humanized” to prevent a patient from mounting an immune response against the antibody when it is used therapeutically. Such antibodies may be sufficiently similar in sequence to human antibodies to be used directly in therapy or may require alteration of a few key residues. Sequence differences between rodent antibodies and human sequences can be minimized by replacing residues which differ from those in the human sequences by site-directed mutagenesis of individual residues or by replacing entire complementarily determining regions. Alternatively, humanized antibodies can be produced using recombinant methods (see, e.g., GB2188638B). Antibodies that specifically bind to a PROK1, PROK2, or GPCR 192 polypeptide can contain antigen binding sites which are either partially or fully humanized, as disclosed in U.S. Pat. No. 5,565,332.

Alternatively, techniques described for the production of single chain antibodies can be adapted using methods known in the art to produce single chain antibodies that specifically bind to PROK1, PROK2, or GPCR 192 polypeptides. Antibodies with related specificity, but of distinct idiotypic composition, can be generated by chain shuffling from random combinatorial immunoglobin libraries (Burton, Proc. Natl. Acad. Sci. 88:11170-23, 1991).

Single-chain antibodies also can be constructed using a DNA amplification method, such as PCR, using hybridoma cDNA as a template (Thirion et al., 1996, Eur J. Cancer Prev. 5:507-11). Single-chain antibodies can be mono- or bispecific, and can be bivalent or tetravalent. Construction of tetravalent, bispecific single-chain antibodies is taught, for example, in Coloma and Morrison, (Nat. Biotechnol. 15:159-63, 1997). Construction of bivalent, bispecific single-chain antibodies is taught in Mallender and Voss, (J. Biol. Chem. 269:199-206, 1994).

A nucleotide sequence encoding a single-chain antibody can be constructed using manual or automated nucleotide synthesis, cloned into an expression construct using standard recombinant DNA methods, and introduced into a cell to express the coding sequence, as described below.

Alternatively, single-chain antibodies can be produced directly using, for example, filamentous phage technology (Verhaar et al., Intl. J. Cancer 61:497-501, 1995; Nicholls et al., J. Immunol. Meth. 165:S1-91, 1993).

Antibodies which specifically bind to PROK1, PROK2, or GPCR 192 polypeptides also can be produced by inducing in vivo production in the lymphocyte population or by screening immunoglobulin libraries or panels of highly specific binding reagents as disclosed in the literature (Orlandi et al., Proc. Natl. Acad. Sci. 8: 3833-3S37, 1989; Winter et al., Nature 349:293-299, 1991).

Other types of antibodies can be constructed and used therapeutically in methods of the invention. For example, chimeric antibodies can be constructed as disclosed in WO 93/03151.

Binding proteins which are derived from immunoglobulins and which are multivalent and multispecific, such as the “diabodies” described in WO 94/13804, also can be prepared.

Antibodies according to the invention can be purified by methods well known in the art. For example, antibodies can be affinity purified by passage over a column to which a PROK1, PROK2, or GPCR 192 polypeptide is bound. The bound antibodies can then be eluted from the column using a buffer with a high salt concentration.

Antisense Oligonucleotides

Antisense oligonucleotides are nucleotide sequences that are complementary to a specific DNA or RNA sequence. Once introduced into a cell, the complementary nucleotides combine with natural sequences produced by the cell to form complexes and block either transcription or translation. Preferably, an antisense oligonucleotide is at least 11 nucleotides in length, but can be at least 12, 15, 20, 25, 30, 35, 40, 45, or 50 or more nucleotides long.

Longer sequences also can be used. Antisense oligonucleotide molecules can be provided in a DNA construct and introduced into a cell as described above to decrease the level of PROK1, PROK2, or GPCR 192 gene products in the cell.

Antisense oligonucleotides can be deoxyribonucleotides, ribonucleotides, or a combination of both. Oligonucleotides can be synthesized manually or by an automated synthesizer, by covalently linking the 5′ end of one nucleotide with the 3′ end of another nucleotide with non-phosphodiester internucleotide linkages such alkylphosphonates, phosphorothioates, phosphorodithioates, allylphosphonothioates, alkylphosphonates, phosphoramidates, phosphate esters, carbamates, acetamidate, carboxymethyl esters, carbonates, and phosphate triesters (see, e.g., Brown, Meth. Mol. Biol. 20:1-8, 1994; Sonveaux, Meth. Mol. Biol. 96:1-72, 1994; Uhlmann et al., Chem. Rev. 90:543-583, 1990.

Modifications of PROK1, PROK2, or GPCR 192 gene expression can be obtained by designing antisense oligonucleotides that will form duplexes to the control, 5′, or regulatory regions of the PROK1, PROK2, or GPCR 192 gene. Oligonucleotides derived from the transcription initiation site, for example, between positions −10 and +10 from the start site, are preferred. Similarly, inhibition can be achieved using “triple helix” base-pairing methodology. Triple helix pairing is useful because it causes inhibition of the ability of the double helix to open sufficiently for the binding of polymerases, transcription factors, or chaperons. Therapeutic advances using triplex DNA have been described in the literature (e.g., Gee et al., in Huber & Carr, MOLECULAR AND IMMUNOLOGIC APPROACHES, Futura Publishing Co., Mt. Kisco, N.Y., 1994). An antisense oligonucleotide also can be designed to block translation of mRNA by preventing the transcript from binding to ribosomes. Precise complementarily is not required for successful complex formation between an antisense oligonucleotide and the complementary sequence of a PROK1, PROK2, or GPCR 192 polynucleotide.

Antisense oligonucleotides which comprise, for example, 2, 3, 4, or 5 or more stretches of contiguous nucleotides which are precisely complementary to a PROK1, PROK2, or GPCR 192 polynucleotide, each separated by a stretch of contiguous nucleotides which are not complementary to adjacent PROK1, PROK2, or GPCR 192 nucleotides, can provide sufficient targeting specificity for PROK1, PROK2, or GPCR 192 mRNA.

Preferably, each stretch of complementary contiguous nucleotides is at least 4, 5, 6, 7, or 8 or more nucleotides in length. Non-complementary intervening sequences are preferably 1, 2, 3, or 4 nucleotides in length. One skilled in the art can easily use the calculated melting point of an antisense-sense pair to determine the degree of mismatching which will be tolerated between a particular antisense oligonucleotide and a particular PROK1, PROK2, or GPCR 192 polynucleotide sequence.

Antisense oligonucleotides can be modified without affecting their ability to hybridize to a PROK1, PROK2, or GPCR 192 polynucleotide. These modifications can be internal or at one or both ends of the antisense molecule. For example, internucleoside phosphate linkages can be modified by adding cholesterol or diamine moieties with varying numbers of carbon residues between the amino groups and terminal ribose. Modified bases and/or sugars, such as arabinose instead of ribose, or a 3′,5′-substituted oligonucleotide in which the 3′ hydroxyl group or the 5′ phosphate group are substituted, also can be employed in a modified antisense oligonucleotide.

These modified oligonucleotides can be prepared by methods well known in the art (see, e.g., Agrawal et al., Trends Biotechnol. 10:152-158, 1992; Uhlmann et al., Chem. Rev. 90:543-584, 1990; Uhlmann et al., Tetrahedrons. Lett. 215:3539-3542, 1987.

Ribozymes.

Ribozymes are RNA molecules with catalytic activity (see, e.g., Cech, Science 236:1532-1539; 1987; Cech, Ann. Rev. Biochem. 59:543-568; 1990, Cech, Curr. Opin. Struct. Biol. 2:605-609, 1992, Couture and Stinchcomb, Trends Tenet. 1:510-515, 1996). Ribozymes can be used to inhibit gene function by cleaving an RNA sequence, as is known in the art (e.g., U.S. Pat. No. 5,641,673). The mechanism of ribozyme action involves sequence-specific hybridization of the ribozyme molecule to complementary target RNA, followed by endonucleolytic cleavage. Examples include engineered hammerhead motif ribozyme molecules that can specifically and efficiently catalyze endonucleolytic cleavage of specific nucleotide sequences.

The coding sequence of a PROK1, PROK2, or GPCR 192 polynucleotide can be used to generate ribozymes that will specifically bind to mRNA transcribed from the PROK1, PROK2, or GPCR 192 polynucleotide. Methods of designing and constructing ribozymes which can cleave other RNA molecules in trans in a highly sequence specific manner have been developed and described in the art (see, e.g., Haseloff et al., Nature 334:585-591, 1988). For example, the cleavage activity of ribozymes can be targeted to specific RNAs by engineering a discrete “hybridization” region into the ribozyme. The hybridization region contains a sequence complementary to the target RNA and thus specifically hybridizes with the target (see, e.g., EP 321,201).

Specific ribozyme cleavage sites within a PROK1, PROK2, or GPCR 192 RNA target can be identified by scanning the target molecule for ribozyme cleavage sites which include the following sequences: GUA, GUU, and GUC. Once identified, short RNA sequences of between 15 and 20 ribonucleotides corresponding to the region of the target RNA containing the cleavage site can be evaluated for secondary structural features which may render the target inoperable.

Suitability of candidate PROK1, PROK2, or GPCR 192 RNA targets also can be evaluated by testing accessibility to hybridization with complementary oligonucleotides using ribonuclease protection assays.

Longer complementary sequences can be used to increase the affinity of the hybridization sequence for the target. The hybridizing and cleavage regions of the ribozyme can be integrally related such that upon hybridizing to the target RNA through the complementary regions, the catalytic region of the ribozyme can cleave the target.

Ribozymes can be introduced into cells as part of a DNA construct. Mechanical methods, such as microinjection, liposome-mediated transfection, electroporation, or calcium phosphate precipitation, can be used to introduce a ribozyme-containing DNA construct into cells in which it is desired to decrease PROK1, PROK2, or GPCR 192 expression. Alternatively, if it is desired that the cells stably retain the DNA construct, the construct can be supplied on a plasmid and maintained as a separate element or integrated into the genome of the cells, as is known in the art. A ribozyme-encoding DNA construct can include transcriptional regulatory elements, such as a promoter element, an enhancer or UAS element, and a transcriptional terminator signal, for controlling transcription of ribozymes in the cells.

As taught in Haseloff et al., (U.S. Pat. No. 5,641,673), ribozymes can be engineered so that ribozyme expression will occur in response to factors that induce expression of a target gene. Ribozymes also can be engineered to provide an additional level of regulation, so that destruction of mRNA occurs only when both a ribozyme and a target gene are induced in the cells.

Differentially Expressed Genes.

Described herein are methods for the identification of genes whose products interact with PROK1, PROK2, or GPCR 192. Such genes may represent genes that are differentially expressed in disorders including, but not limited to, central nervous system disorders, immune disorders, metabolic disorders, or cancer. Further, such genes may represent genes that are differentially regulated in response to manipulations relevant to the progression or treatment of such diseases. Additionally, such genes may have a temporally modulated expression, increased or decreased at different stages of tissue or organism development. A differentially expressed gene may also have its expression modulated under control versus experimental conditions. In addition, PROK1, PROK2, or GPCR 192 gene or gene product may itself be tested for differential expression.

The degree to which expression differs in a normal versus a diseased state need only be large enough to be visualized via standard characterization techniques such as differential display techniques. Other such standard characterization techniques by which expression differences may be visualized include but are not limited to, quantitative RT (reverse transcriptase), PCR, and Northern analysis.

Identification of Differentially Expressed Genes

To identify differentially expressed genes, total RNA or, preferably, mRNA is isolated from tissues of interest. For example, RNA samples are obtained from tissues of experimental subjects and from corresponding tissues of control subjects. Any RNA isolation technique that does not select against the isolation of mRNA may be utilized for the purification of such RNA samples (see, e.g., Ausubel et al., ed., CURRENT PROTOCOLS IN MOLECULAR BIOLOGY, John Wiley & Sons, Inc. New York, 1987-1993). Large numbers of tissue samples may readily be processed using techniques well known to those of skill in the art, such as, for example, the single-step RNA isolation process of Chomczynski, (U.S. Pat. No. 4,843,155).

Transcripts within the collected RNA samples that represent RNA produced by differentially expressed genes are identified by methods well known to those of skill in the art. They include, for example, differential screening (Tedder et al., Proc. Natl. Acad. Sci. U.S.A. 85:708-12, 1988), subtractive hybridization (Hedrick et al., Nature 308:149-53; Lee et al., Proc. Natl. Acad. Sci. U.S.A. 88:2825, 1984), and, preferably, differential display (Liang and Pardee, Science 257:967-71, 1992; U.S. Pat. No. 5,262,311).

The differential expression information may itself suggest relevant methods for the treatment of disorders involving PROK1, PROK2, or GPCR 192. For example, treatment may include a modulation of expression of the differentially expressed genes and/or the gene encoding PROK1, PROK2, or GPCR 192. The differential expression information may indicate whether the expression or activity of the differentially expressed gene or gene product or the PROK1, PROK2, or GPCR 192 gene or gene product are up-regulated or down-regulated.

Test Compounds.

Test compounds can be pharmacologic agents already known in the art or can be compounds previously unknown to have any pharmacological activity. The compounds can be naturally occurring or designed in the laboratory. They can be isolated from microorganisms, animals, or plants, and can be produced recombinantly, or synthesized by chemical methods known in the art. If desired, test compounds can be obtained using any of the numerous combinatorial library methods known in the art, including but not limited to, biological libraries, spatially addressable parallel solid phase or solution phase libraries, synthetic library methods requiring deconvolution, the “one-bead one-compound” library method, and synthetic library methods using affinity chromatography selection. The biological library approach is limited to polypeptide libraries, while the other four approaches are applicable to polypeptide, non-peptide oligomer, or small molecule libraries of compounds (Lam, Anticancer Drug Des. 12:145, 1997).

Methods for the synthesis of molecular libraries are well known in the art (see, e.g., DeWitt et al., Proc. Natl. Acad. Sci. U.S.A. 90:6909, 1993; Erb et al., Proc. Natl. Acad. Sci. U.S.A. 91:11422, 1994; Zuckermann et al., J. Med. Chem. 37:2678, 1994; Cho et al., Science 261:1303, 1993; Carell et al., Angew. Chem. Int. Ed. Engl. 33:2059, 1994; Carell et al., Angew. Chem. Int. Ed. Engl. 33:2061; Gallop et al., J. Med. Chem. 37:1233, 1994).

Libraries of compounds can be presented in solution (see, e.g., Houghten, BioTechniques 13:412-421, 1992), or on beads (Lam, Nature 354:82-84, 1991), chips (Fodor, Nature 364:555-556, 1993), bacteria or spores (U.S. Pat. No. 5,223,409), plasmids (Cull et al., Proc. Natl. Acad. Sci. U.S.A. 89:1865-1869, 1992), or phage (Scott and Smith, Science 249:386-390, 1990; Devlin, Science 249:404-406, 1990; Cwirla et al., Proc. Natl. Acad. Sci. U.S.A. 97:6378-6382, 1990; Felici, J. Mol. Biol. 22: 301-310, 1991; and U.S. Pat. No. 5,223,409).

High Throughput Screening.

Test compounds can be screened for the ability to affect the binding of PROK1 or PROK2 polypeptides to GPCR polypeptides, or to affect PROK1, PROK2, or GPCR 192 activity, or PROK1, PROK2, or GPCR 192 gene expression using high throughput screening. Using high throughput screening, many discrete compounds can be tested in parallel so that large numbers of test compounds can be quickly screened. The most widely established techniques utilize 96-well microtiter plates. The wells of the microtiter plates typically require assay volumes that range from 50 to 500 μl. In addition to the plates, many instruments, materials, pipettors, robotics, plate washers, and plate readers are commercially available to fit the 96-well format.

Alternatively, “free format assays,” or assays that have no physical barrier between samples, can be used. For example, an assay using pigment cells (melanocytes) in a simple homogeneous assay for combinatorial peptide libraries is described by Jayawickreme et al., (Proc. Natl. Acad. Sci. U.S.A. 19:1614-18, 1994). The cells are placed under agarose in petri dishes, then beads that carry combinatorial compounds are placed on the surface of the agarose.

The combinatorial compounds are partially released the compounds from the beads. Active compounds can be visualized as dark pigment areas because, as the compounds diffuse locally into the gel matrix, the active compounds cause the cells to change colors.

Another example of a free format assay is described by Chelsky, (“Strategies for Screening Combinatorial Libraries: Novel and Traditional Approaches,” reported at the First Annual Conference of The Society for Biomolecular Screening in Philadelphia, Pa., Nov. 7-10, 1995). Chelsky placed a simple homogenous enzyme assay for carbonic anhydrase inside an agarose gel such that the enzyme in the gel would cause a color change throughout the gel.

Thereafter, beads carrying combinatorial compounds via a photolinker were placed inside the gel and the compounds were partially released by W-light. Compounds that inhibited the enzyme were observed as local zones of inhibition having less color change.

Yet another example is described by Salmon et al., (Molecul. Diversity 2:57-63, 1996). In this example, combinatorial libraries were screened for compounds that had cytotoxic effects on cancer cells growing in agar.

Another high throughput screening method is described in Beutel et al., (U.S. Pat. No. 5,976,813). In this method, test samples are placed in a porous matrix. One or more assay components are then placed within, on top of, or at the bottom of a matrix such as a gel, a plastic sheet, a filter, or other form of easily manipulated solid support. When samples are introduced to the porous matrix they diffuse sufficiently slowly, such that the assays can be performed without the test samples running together.

Binding Assays.

For binding assays, the test compound is preferably a small molecule that binds to and occupies, for example, the binding domain of the GPCR polypeptide, such that the enzymatic activity is inhibited. Examples of such small molecules include, but are not limited to, small peptides or peptide-like molecules.

In binding assays, either the test compound or the PROK1 or PROK2 polypeptide can comprise a detectable label, such as a fluorescent, radioisotopic, chemiluminescent, or enzymatic label, such as horseradish peroxidase, alkaline phosphatase, or luciferase. Detection of a test compound that is bound to the GPCR 192 polypeptide or alters the ligand binding activity of PROK1 or PROK2 can then be accomplished, for example, by direct counting of radioemmission, by scintillation counting, or by determining conversion of an appropriate substrate to a detectable product.

It may be desirable to immobilize either the GPCR 192 polypeptide or polynucleotide or the test compound, PROK1, or PROK2 to facilitate separation of bound from unbound forms of one or both of the interactants, as well as to accommodate automation of the assay. Thus, either the PROK1 or PROK2 polypeptide or polynucleotide, the test compound, or the GPCR 192 polypeptide or polynucleotide can be bound to a solid support. Suitable solid supports include, but are not limited to, glass or plastic slides, tissue culture plates, microtiter wells, tubes, silicon chips, or particles such as beads including, but not limited to, latex, polystyrene, or glass beads). Any method known in the art can be used to attach the polypeptide or polynucleotide or test compound to a solid support, including use of covalent and non-covalent linkages, passive absorption, or pairs of binding moieties attached respectively to the polypeptide or polynucleotide or test compound and the solid support. Test compounds are preferably bound to the solid support in an array, so that the location of individual test compounds can be tracked. Binding of a test compound to a GPCR 192 polypeptide or polynucleotide can be accomplished in any vessel suitable for containing the reactants.

Examples of such vessels include microtiter plates, test tubes, and microcentrifuge tubes. In one embodiment, the GPCR 192 polypeptide is a fusion protein comprising a domain that allows the GPCR 192 polypeptide to be bound to a solid support. For example, glutathione-S-transferase fusion proteins can be adsorbed onto glutathione sepharose beads (Sigma Chemical, St. Louis, Mo.) or glutathione-derivatized microtiter plates, which are then combined with the test compound or the test compound and the PROK1 or PROK2 polypeptide; the mixture is then incubated under conditions conducive to complex formation (e.g., at physiological conditions for salt and pH). Following incubation, the beads or microtiter plate wells are washed to remove any unbound components. Binding of the interactants can be determined either directly or indirectly, as described above. Alternatively, the complexes can be dissociated from the solid support before binding is determined.

Other techniques for immobilizing proteins or polynucleotides on a solid support also can be used in the screening assays of the invention. For example, either a PROK1 or PROK2 polypeptide or polynucleotide or a test compound can be immobilized utilizing conjugation of biotin and streptavidin. Biotinylated PROK1 or PROK2 polypeptides or polynucleotides or test compounds can be prepared from biotin-NHS(N-hydroxysuccinimide) using techniques well known in the art (e.g., biotinylation kit, Pierce Chemicals, Rockford, Ill.) and immobilized in the wells of streptavidin-coated 96-well plates (Pierce Chemical). Alternatively, antibodies which specifically bind to a PROK1 or PROK2 polypeptide, polynucleotide, or a test compound, but which do not interfere with a desired binding site, such as the active site of the GPCR 192 polypeptide, can be derivatized to the wells of the plate. Unbound target or protein can be trapped in the wells by antibody conjugation.

Methods for detecting such complexes, in addition to those described above for the GST-immobilized complexes, include immunodetection of complexes using antibodies which specifically bind to GPCR 192, PROK1, PROK2 polypeptide, or test compound, enzyme-linked assays, and SDS gel electrophoresis under non-reducing conditions.

Screening for test compounds which bind to GPCR 192 polypeptide or polynucleotide also can be carried out in an intact cell. Any cell which comprises a GPCR 192 polypeptide or polynucleotide can be used in a cell-based assay system. A GPCR 192 polynucleotide can be naturally occurring in the cell or can be introduced using techniques such as those described above. Binding of the test compound to a GPCR 192 polypeptide or polynucleotide is determined as described above.

In yet another aspect of the invention, a PROK1, PROK2, or GPCR 192 polypeptide can be used as a “bait protein” in a two-hybrid assay or three-hybrid assay (see, e.g., U.S. Pat. No. 5,283,317; Zervos et al., Cell 72:223-232, 1993; Madura et al., J. Biol. Chem. 68:12046-12054, 1993; Bartel et al., BioTechniques 14:990-924, 1993; Iwabuchi et al., Oncogene 8:1693-1696, 1993; and WO94/10300), to identify other proteins which bind to or interact with PROK1, PROK2, or GPCR 192 polypeptide and modulate its activity.

The two-hybrid system is based on the modular nature of most transcription factors, which consist of separable DNA-binding and activation domains. Briefly, the assay utilizes two different DNA constructs. For example, in one construct, polynucleotide encoding PROK1, PROK2, or GPCR 192 polypeptide can be fused to a polynucleotide encoding the DNA binding domain of a known transcription factor (e.g., GAL4). In the other construct, a DNA sequence that encodes an unidentified protein (“prey” or “sample”) can be fused to a polynucleotide that codes for the activation domain of the known transcription factor. If the “bait” and the “prey” proteins are able to interact in vivo to form an protein-dependent complex, the DNA-binding and activation domains of the transcription factor are brought into close proximity. This proximity allows transcription of a reporter gene (e.g., LacZ), which is operably linked to a transcriptional regulatory site responsive to the transcription factor. Expression of the reporter gene can be detected, and cell colonies containing the functional transcription factor can be isolated and used to obtain the DNA sequence encoding the protein that interacts with PROK1, PROK2, or GPCR 192 polypeptide.

Gene Expression.

In another embodiment, test compounds that increase or decrease PROK1, PROK2, or GPCR 192 gene expression are identified. A PROK1, PROK2, or GPCR 192 polynucleotide is contacted with a test compound, and the expression of an RNA or polypeptide product of the PROK1, PROK2, or GPCR 192 polynucleotide is determined. The level of expression of appropriate mRNA or polypeptide in the presence of the test compound is compared to the level of expression of mRNA or polypeptide in the absence of the test compound. The test compound can then be identified as a modulator of expression based on this comparison. For example, when expression of mRNA or polypeptide is greater in the presence of the test compound than in its absence, the test compound is identified as a stimulator or enhancer of the mRNA or polypeptide expression. Alternatively, when expression of the mRNA or polypeptide is less in the presence of the test compound than in its absence, the test compound is identified as an inhibitor of the mRNA or polypeptide expression. The level of PROK1, PROK2, or GPCR 192 mRNA or polypeptide expression in the cells can be determined by methods well known in the art for detecting mRNA or polypeptide. Either qualitative or quantitative methods can be used. The presence of polypeptide products of PROK1, PROK2, or GPCR 192 polynucleotide can be determined, for example, using a variety of techniques known in the art, including immunochemical methods such as radioimmunoassay, Western blotting, and immunohistochemistry. Alternatively, polypeptide synthesis can be determined in vivo, in a cell culture, or in an in vitro translation system by detecting incorporation of labeled amino acids into PROK1, PROK2, or GPCR 192 polypeptide. Such screening can be carried out either in a cell-free assay system or in an intact cell.

Any cell that expresses PROK1, PROK2, or GPCR 192 polynucleotide can be used in a cell-based assay system. The PROK1, PROK2, or GPCR 192 polynucleotide can be naturally occurring in the cell or can be introduced using techniques such as those described above. Either a primary culture or an established cell line, such as CHO or human embryonic kidney 293 cells, can be used.

Diagnostic Methods

PROK1, PROK2, or GPCR 192 also can be used in diagnostic assays for detecting diseases and abnormalities or susceptibility to diseases and abnormalities related to the presence of mutations in the nucleic acid sequences that encode PROK1, PROK2, or GPCR 192. For example, differences can be determined between the cDNA or genomic sequence encoding PROK1, PROK2, or GPCR 192 in individuals afflicted with a disease and in normal individuals. If a mutation is observed in some or all of the afflicted individuals but not in normal individuals, then the mutation is likely to be the causative agent of the disease.

Sequence differences between a reference gene and a gene having mutations can be revealed by the direct DNA sequencing method. In addition, cloned DNA segments can be employed as probes to detect specific DNA segments. The sensitivity of this method is greatly enhanced when combined with PCR. For example, a sequencing primer can be used with a double-stranded PCR product or a single-stranded template molecule generated by a modified PCR. The sequence determination is performed by conventional procedures using radiolabeled nucleotides or by automatic sequencing procedures using fluorescent tags.

Genetic testing based on DNA sequence differences can be carried out by detection of alteration in electrophoretic mobility of DNA fragments in gels with or without denaturing agents. Small sequence deletions and insertions can be visualized, for example, by high resolution gel electrophoresis. DNA fragments of different sequences can be distinguished on denaturing formamide gradient gels in which the mobilities of different DNA fragments are retarded in the gel at different positions according to their specific melting or partial melting temperatures (see, e.g., Myers et al., Science 230:1242, 1985). Sequence changes at specific locations can also be revealed by nuclease protection assays, such as RNase and S1 protection or the chemical cleavage method (e.g., Cotton et al., Proc. Natl. Acad. Sci. USA 85:4397-4401, 1985). Thus, the detection of a specific DNA sequence can be performed by methods such as hybridization, RNase protection, chemical cleavage, direct DNA sequencing or the use of restriction enzymes and Southern blotting of genomic DNA. In addition to direct methods such as gel-electrophoresis and DNA sequencing, mutations can also be detected by in situ analysis.

Altered levels of PROK1, PROK2, or GPCR 192 also can be detected in various tissues. Assays used to detect levels of PROK1, PROK2, or GPCR 192 polypeptides in a body sample, such as blood or a tissue biopsy, derived from a host are well known to those of skill in the art and include radioimmunoassays, competitive binding assays, Western blot analysis, and ELISA assays.

Therapeutic Indications and Methods.

This invention further pertains to the use of novel agents identified by the screening assays described above. Accordingly, it is within the scope of this invention to use a test compound identified as described herein in an appropriate animal model. For example, an agent identified as described herein (e.g., a modulating agent, an antisense nucleic acid molecule, a specific antibody, ribozyme) can be used in an animal model to determine the efficacy, toxicity, or side effects of treatment with such an agent. Alternatively, an agent identified as described herein can be used in an animal model to determine the mechanism of action of such an agent. Furthermore, this invention pertains to uses of novel agents identified by the above-described screening assays for treatments as described herein.

A reagent which affects PROK1, PROK2, or GPCR 192 activity can be administered to a human or animal cell, either in vitro or in vivo, to alter PROK1, PROK2, or GPCR 192 activity. The reagent preferably binds to an expression product of a human PROK1, PROK2, or GPCR 192 gene. If the expression product is a protein, the reagent is preferably an antibody. For treatment of human cells ex vivo, an antibody can be added to a preparation of stem cells that have been removed from the body. The cells can then be replaced in the same or another human body, with or without clonal propagation, as is known in the art.

In one embodiment, the reagent is delivered using a liposome. Preferably, the liposome is stable in the animal into which it has been administered for at least about 30 minutes, more preferably for at least about 1 hour, and even more preferably for at least about 24 hours. A liposome comprises a lipid composition that is capable of targeting a reagent, particularly a polynucleotide, to a particular site in an animal, such as a human. Preferably, the lipid composition of the liposome is capable of targeting to a specific organ of an animal, such as the lung, liver, spleen, heart brain, lymph nodes, and skin.

A liposome useful in the present invention comprises a lipid composition that is capable of fusing with the plasma membrane of the targeted cell to deliver its contents to the cell. Preferably, the transfection efficiency of a liposome is about 0.5 μg of DNA per 16 nmole of liposome delivered to about 10⁶ cells, more preferably about 1.0 μg of DNA per 16 nmole of liposome delivered to about 10⁶ cells, and even more preferably about 2.0 μg of DNA per 16 nmol of liposome delivered to about 10⁶ cells. Preferably, a liposome is between about 100 and 500 nm, more preferably between about 150 and 450 nm, and even more preferably between about 200 and 400 nm in diameter.

Suitable liposomes for use in the present invention include those liposomes standardly used in, for example, gene delivery methods known to those of skill in the art. More preferred liposomes include liposomes having a polycationic lipid composition and/or liposomes having a cholesterol backbone conjugated to polyethylene glycol. Optionally, a liposome comprises a compound capable of targeting the liposome to a particular cell type, such as a cell-specific ligand exposed on the outer surface of the liposome.

Complexing a liposome with a reagent such as an antisense oligonucleotide or ribozyme can be achieved using methods that are standard in the art (see, e.g., U.S. Pat. No. 5,705,151). Preferably, from about 0.1 μg to about 10 μg of polynucleotide is combined with about 8 nmol of liposomes, more preferably from about 0.5 μg to about 5 μg of polynucleotides are combined with about 8 mol liposomes, and even more preferably about 1.0 μg of polynucleotides is combined with about 8 mol liposomes.

In another embodiment, antibodies can be delivered to specific tissues in vivo using receptor-mediated targeted delivery. Receptor-mediated DNA delivery techniques are taught in, for example, Findeis et al., (Trends in Biotechnol. 11:202-05, 1993); Chiou et al., (GENE THERAPEUTICS: METHODS AND APPLICATIONS OF DIRECT GENE TRANSFER, J. A. Wolff, ed., 1994); Wu and Wu, (J. Biol. Chem. 263:621-24, 1988); Wu et al., (J. Biol. Chem. 269:542-46, 1994); Zenke et al., (Proc. Natl. Acad. Sci. U.S.A. 87:3655-59, 1990); Wu et al., (J. Biol. Chem. 66:338-42, 1991).

Pharmaceutical Compositions

The invention also provides pharmaceutical compositions that can be administered to a patient to achieve a therapeutic effect. Pharmaceutical compositions of the invention can comprise, for example, PROK1, PROK2, or GPCR 192 polypeptide, PROK1, PROK2, or GPCR 192 polynucleotide, ribozymes or antisense oligonucleotides, antibodies which specifically bind to PROK1, PROK2, or GPCR 192 polypeptide, or mimetics, activators, or inhibitors of PROK1, PROK2, or GPCR 192 polypeptide activity. The compositions can be administered to a patient alone, or in combination with other agents, drugs or hormones.

Based on the assays described herein, or other well known assays used to evaluate activation or inhibition of a receptor site by a compound, by standard toxicity tests, and by standard pharmacological assays for the determination of treatment of GPCR 192 receptor-mediated conditions identified above in mammals, and by comparison of these results with the results of known medicaments that are used to treat these conditions, the effective dosage of compounds identified by the methods of this invention can readily be determined for treatment of each desired indication. The amount of the active ingredient to be administered in the treatment of one of these conditions can vary widely according to such considerations as the particular compound and dosage unit employed, the mode of administration, the period of treatment, the age and sex of the patient treated, and the nature and extent of the condition treated.

The total amount of the active ingredient to be administered may generally range from about 0.001 mg/kg to about 200 mg/kg, and preferably from about 0.01 mg/kg to about 200 mg/kg body weight per day. A unit dosage may contain from about 0.05 mg to about 1500 mg of active ingredient, and may be administered one or more times per day. The daily dosage for administration by injection, including intravenous, intramuscular, subcutaneous, and parenteral injections, and use of infusion techniques may be from about 0.01 to about 200 mg/kg. The daily rectal dosage regimen may be from 0.01 to 200 mg/kg of total body weight. The transdermal concentration may be that required to maintain a daily dose of from 0.01 to 200 mg/kg.

Of course, the specific initial and continuing dosage regimen for each patient will vary according to the nature and severity of the condition as determined by the attending diagnostician, the activity of the specific compound employed, the age of the patient, the diet of the patient, time of administration, route of administration, rate of excretion of the drug, drug combinations, and the like. The desired mode of treatment and number of doses of a compound of the present invention or a pharmaceutically acceptable salt thereof may be ascertained by those skilled in the art using conventional treatment tests.

The compounds identified by the methods of this invention may be utilized to achieve the desired pharmacological effect by administration to a patient in need thereof in an appropriately formulated pharmaceutical composition. A patient, for the purpose of this invention, is a mammal, including, but not limited to, humans, monkeys, dogs, cats, cows, horses, rabbits, in need of treatment for a particular GPCR 192 receptor-mediated condition or disease. Therefore, the present invention includes pharmaceutical compositions which are comprised of a pharmaceutically acceptable carrier and a pharmaceutically effective amount of a compound identified by the methods described herein, or a pharmaceutically acceptable salt or ester thereof. A pharmaceutically acceptable carrier is any carrier which is relatively non-toxic and innocuous to a patient at concentrations consistent with effective activity of the active ingredient so that any side effects ascribable to the carrier do not vitiate the beneficial effects of the active ingredient. A pharmaceutically effective amount of a compound is that amount which produces a result or exerts an influence on the particular condition being treated. The compounds identified by the methods described herein may be administered with a pharmaceutically-acceptable carrier using any effective conventional dosage unit forms, including, for example, immediate and timed release preparations, orally, parenterally, topically, or the like.

For oral administration, the compounds may be formulated into solid or liquid preparations such as, for example, capsules, pills, tablets, troches, lozenges, melts, powders, solutions, suspensions, or emulsions, and may be prepared according to methods known to the art for the manufacture of pharmaceutical compositions. The solid unit dosage forms may be a capsule which can be of the ordinary hard- or soft-shelled gelatin type containing, for example, surfactants, lubricants, and inert fillers such as lactose, sucrose, calcium phosphate, and corn starch.

In another embodiment, the compounds of this invention may be tableted with conventional tablet bases such as lactose, sucrose, and cornstarch in combination with binders such as acacia, cornstarch, or gelatin; disintegrating agents intended to assist the break-up and dissolution of the tablet following administration such as potato starch, alginic acid, corn starch, and guar gum; lubricants intended to improve the flow of tablet granulation and to prevent the adhesion of tablet material to the surfaces of the tablet dies and punches, for example, talc, stearic acid, or magnesium, calcium or zinc stearate; dyes; coloring agents; and flavoring agents intended to enhance the aesthetic qualities of the tablets and make them more acceptable to the patient. Suitable excipients for use in oral liquid dosage forms include diluents such as water and alcohols, for example, ethanol, benzyl alcohol, and polyethylene alcohols, either with or without the addition of a pharmaceutically acceptable surfactant, suspending agent, or emulsifying agent. Various other materials may be present as coatings or to otherwise modify the physical form of the dosage unit. For instance tablets, pills or capsules may be coated with shellac, sugar or both.

Dispersible powders and granules are suitable for the preparation of an aqueous suspension. They provide the active ingredient in admixture with a dispersing or wetting agent, a suspending agent, and one or more preservatives. Suitable dispersing or wetting agents and suspending agents are exemplified by those already mentioned above. Additional excipients, for example, those sweetening, flavoring and coloring agents described above, may also be present.

The pharmaceutical compositions of this invention may also be in the form of oil-in-water emulsions. The oily phase may be a vegetable oil such as liquid paraffin or a mixture of vegetable oils. Suitable emulsifying agents may be (1) naturally occurring gums such as gum acacia and gum tragacanth, (2) naturally occurring phosphatides such as soy bean and lecithin, (3) esters or partial esters derived from fatty acids and hexitol anhydrides, for example, sorbitan monooleate, and (4) condensation products of said partial esters with ethylene oxide, for example, polyoxyethylene sorbitan monooleate. The emulsions may also contain sweetening and flavoring agents.

Oily suspensions may be formulated by suspending the active ingredient in a vegetable oil such as, for example, arachis oil, olive oil, sesame oil, or coconut oil; or in a mineral oil such as liquid paraffin. The oily suspensions may contain a thickening agent such as, for example, beeswax, hard paraffin, or cetyl alcohol. The suspensions may also contain one or more preservatives, for example, ethyl or n-propyl p-hydroxybenzoate; one or more coloring agents; one or more flavoring agents; and one or more sweetening agents such as sucrose or saccharin.

Syrups and elixirs may be formulated with sweetening agents such as, for example, glycerol, propylene glycol, sorbitol, or sucrose. Such formulations may also contain a demulcent, and preservative, flavoring and coloring agents.

The compounds identified by the methods of this invention may also be administered parenterally, that is, subcutaneously, intravenously, intramuscularly, or interperitoneally, as injectable dosages of the compound in a physiologically acceptable diluent with a pharmaceutical carrier which may be a sterile liquid or mixture of liquids such as water, saline, aqueous dextrose and related sugar solutions; an alcohol such as ethanol, isopropanol, or hexadecyl alcohol; glycols such as propylene glycol or polyethylene glycol; glycerol ketals such as 2,2-dimethyl-1,1-dioxolane-4-methanol, ethers such as poly(ethyleneglycol) 400; an oil; a fatty acid; a fatty acid ester or glyceride; or an acetylated fatty acid glyceride with or without the addition of a pharmaceutically acceptable surfactant such as a soap or a detergent, suspending agent such as pectin, carbomers, methycellulose, hydroxypropylmethylcellulose, or carboxymethylcellulose, or emulsifying agent and other pharmaceutical adjuvants.

Illustrative of oils which can be used in the parenteral formulations of this invention are those of petroleum, animal, vegetable, or synthetic origin, for example, peanut oil, soybean oil, sesame oil, cottonseed oil, corn oil, olive oil, petrolatum, and mineral oil. Suitable fatty acids include oleic acid, stearic acid, and isostearic acid. Suitable fatty acid esters are, for example, ethyl oleate and isopropyl myristate. Suitable soaps include fatty alkali metal, ammonium, and triethanolamine salts and suitable detergents include cationic detergents, for example, dimethyl dialkyl ammonium halides, alkyl pyridinium halides, and alkylamine acetates; anionic detergents, for example, alkyl, aryl, and olefin sulfonates, alkyl, olefin, ether, and monoglyceride sulfates, and sulfosuccinates; nonionic detergents, for example, fatty amine oxides, fatty acid alkanolamides, and polyoxyethylenepolypropylene copolymers; and amphoteric detergents, for example, alkyl-beta-aminopropionates, and 2-alkylimidazoline quarternary ammonium salts, as well as mixtures.

The parenteral compositions of this invention may typically contain from about 0.5% to about 25% by weight of the active ingredient in solution. Preservatives and buffers may also be used advantageously. In order to minimize or eliminate irritation at the site of injection, such compositions may contain a non-ionic surfactant having a hydrophile-lipophile balance (HLB) of from about 12 to about 17. The quantity of surfactant in such formulation ranges from about 5% to about 15% by weight. The surfactant can be a single component having the above HLB or can be a mixture of two or more components having the desired HLB.

Illustrative of surfactants used in parenteral formulations are the class of polyethylene sorbitan fatty acid esters, for example, sorbitan monooleate and the high molecular weight adducts of ethylene oxide with a hydrophobic base, formed by the condensation of propylene oxide with propylene glycol.

The pharmaceutical compositions may be in the form of sterile injectable aqueous suspensions. Such suspensions may be formulated according to known methods using suitable dispersing or wetting agents and suspending agents such as, for example, sodium carboxymethylcellulose, methylcellulose, hydroxypropylmethyl-cellulose, sodium alginate, polyvinylpyrrolidone, gum tragacanth and gum acacia; dispersing or wetting agents which may be a naturally occurring phosphatide such as lecithin, a condensation product of an alkylene oxide with a fatty acid, for example, polyoxyethylene stearate, a condensation product of ethylene oxide with a long chain aliphatic alcohol, for example, heptadecaethyleneoxycetanol, a condensation product of ethylene oxide with a partial ester derived form a fatty acid and a hexitol such as polyoxyethylene sorbitol monooleate, or a condensation product of an ethylene oxide with a partial ester derived from a fatty acid and a hexitol anhydride, for example polyoxyethylene sorbitan monooleate.

The sterile injectable preparation may also be a sterile injectable solution or suspension in a non-toxic parenterally acceptable diluent or solvent. Diluents and solvents that may be employed are, for example, water, Ringer's solution, and isotonic sodium chloride solution. In addition, sterile fixed oils are conventionally employed as solvents or suspending media. For this purpose, any bland, fixed oil may be employed including synthetic mono or diglycerides. In addition, fatty acids such as oleic acid may be used in the preparation of injectables.

A composition of the invention may also be administered in the form of suppositories for rectal administration of the drug. These compositions may be prepared by mixing the drug with a suitable non-irritation excipient which is solid at ordinary temperatures but liquid at the rectal temperature and will therefore melt in the rectum to release the drug. Such material are, for example, cocoa butter and polyethylene glycol.

Another formulation employed in the methods of the present invention employs transdermal delivery devices (“patches”). Such transdermal patches may be used to provide continuous or discontinuous infusion of the compounds of the present invention in controlled amounts. The construction and use of transdermal patches for the delivery of pharmaceutical agents is well known in the art (see, e.g., U.S. Pat. No. 5,023,252, incorporated herein by reference). Such patches may be constructed for continuous, pulsatile, or on demand delivery of pharmaceutical agents.

It may be desirable or necessary to introduce the pharmaceutical composition to the patient via a mechanical delivery device. The construction and use of mechanical delivery devices for the delivery of pharmaceutical agents is well known in the art. For example, direct techniques for administering a drug directly to the brain usually involve placement of a drug delivery catheter into the patient's ventricular system to bypass the blood-brain barrier. One such implantable delivery system, used for the transport of agents to specific anatomical regions of the body, is described in U.S. Pat. No. 5,011,472, incorporated herein by reference.

The compositions of the invention may also contain other conventional pharmaceutically acceptable compounding ingredients, generally referred to as carriers or diluents, as necessary or desired. Any of the compositions of this invention may be preserved by the addition of an antioxidant such as ascorbic acid or by other suitable preservatives. Conventional procedures for preparing such compositions in appropriate dosage forms can be utilized.

Commonly used pharmaceutical ingredients which may be used as appropriate to formulate the composition for its intended route of administration include: acidifying agents, for example, but are not limited to, acetic acid, citric acid, fumaric acid, hydrochloric acid, nitric acid; and alkalinizing agents such as, but are not limited to, ammonia solution, ammonium carbonate, diethanolamine, monoethanolamine, potassium hydroxide, sodium borate, sodium carbonate, sodium hydroxide, triethanolamine, trolamine.

Other pharmaceutical ingredients include, for example, but are not limited to, adsorbents (e.g., powdered cellulose and activated charcoal); aerosol propellants (e.g., carbon dioxide, CCl₂F₂, F₂ClC—CClF₂ and CClF₃); air displacement agents (e.g., nitrogen and argon); antifungal preservatives (e.g., benzoic acid, butylparaben, ethylparaben, methylparaben, propylparaben, sodium benzoate); antimicrobial preservatives (e.g., benzalkonium chloride, benzethonium chloride, benzyl alcohol, cetylpyridinium chloride, chlorobutanol, phenol, phenylethyl alcohol, phenylmercuric nitrate and thimerosal); antioxidants (e.g., ascorbic acid, ascorbyl palmitate, butylated hydroxyanisole, butylated hydroxytoluene, hypophosphorus acid, monothioglycerol, propyl gallate, sodium ascorbate, sodium bisulfite, sodium formaldehyde sulfoxylate, sodium metabisulfite); binding materials (e.g., block polymers, natural and synthetic rubber, polyacrylates, polyurethanes, silicones and styrene-butadiene copolymers); buffering agents (e.g., potassium metaphosphate, potassium phosphate monobasic, sodium acetate, sodium citrate anhydrous and sodium citrate dihydrate); carrying agents (e.g., acacia syrup, aromatic syrup, aromatic elixir, cherry syrup, cocoa syrup, orange syrup, syrup, corn oil, mineral oil, peanut oil, sesame oil, bacteriostatic sodium chloride injection and bacteriostatic water for injection); chelating agents (e.g., edetate disodium and edetic acid); colorants (e.g., FD&C Red No. 3, FD&C Red No. 20, FD&C Yellow No. 6, FD&C Blue No. 2, D&C Green No. 5, D&C Orange No. 5, D&C Red No. 8, caramel and ferric oxide red); clarifying agents (e.g., bentonite); emulsifying agents (but are not limited to, acacia, cetomacrogol, cetyl alcohol, glyceryl monostearate, lecithin, sorbitan monooleate, polyethylene 50 stearate); encapsulating agents (e.g., gelatin and cellulose acetate phthalate); flavorants (e.g., anise oil, cinnamon oil, cocoa, menthol, orange oil, peppermint oil and vanillin); humectants (e.g., glycerin, propylene glycol and sorbitol); levigating agents (e.g., mineral oil and glycerin); oils (e.g., arachis oil, mineral oil, olive oil, peanut oil, sesame oil and vegetable oil); ointment bases (e.g., lanolin, hydrophilic ointment, polyethylene glycol ointment, petrolatum, hydrophilic petrolatum, white ointment, yellow ointment, and rose water ointment); penetration enhancers (transdermal delivery) (e.g., monohydroxy or polyhydroxy alcohols, saturated or unsaturated fatty alcohols, saturated or unsaturated fatty esters, saturated or unsaturated dicarboxylic acids, essential oils, phosphatidyl derivatives, cephalin, terpenes, amides, ethers, ketones and ureas); plasticizers (e.g., diethyl phthalate and glycerin); solvents (e.g., alcohol, corn oil, cottonseed oil, glycerin, isopropyl alcohol, mineral oil, oleic acid, peanut oil, purified water, water for injection, sterile water for injection and sterile water for irrigation); stiffening agents (e.g., cetyl alcohol, cetyl esters wax, microcrystalline wax, paraffin, stearyl alcohol, white wax and yellow wax); suppository bases (e.g., cocoa butter and polyethylene glycols (mixtures)); surfactants (e.g., benzalkonium chloride, nonoxynol 10, oxtoxynol 9, polysorbate 80, sodium lauryl sulfate and sorbitan monopalmitate); suspending agents (e.g., agar, bentonite, carbomers, carboxymethylcellulose sodium, hydroxyethyl cellulose, hydroxypropyl cellulose, hydroxypropyl methylcellulose, kaolin, methylcellulose, tragacanth and veegum); sweetening e.g., aspartame, dextrose, glycerin, mannitol, propylene glycol, saccharin sodium, sorbitol and sucrose); tablet anti-adherents (e.g., magnesium stearate and talc); tablet binders (e.g., acacia, alginic acid, carboxymethylcellulose sodium, compressible sugar, ethylcellulose, gelatin, liquid glucose, methylcellulose, povidone and pregelatinized starch); tablet and capsule diluents (e.g., dibasic calcium phosphate, kaolin, lactose, mannitol, microcrystalline cellulose, powdered cellulose, precipitated calcium carbonate, sodium carbonate, sodium phosphate, sorbitol and starch); tablet coating agents (e.g., liquid glucose, hydroxyethyl cellulose, hydroxypropyl cellulose, hydroxypropyl methylcellulose, methylcellulose, ethylcellulose, cellulose acetate phthalate and shellac); tablet direct compression excipients (e.g., dibasic calcium phosphate); tablet disintegrants (e.g., alginic acid, carboxymethylcellulose calcium, microcrystalline cellulose, polacrillin potassium, sodium alginate, sodium starch glycollate and starch); tablet glidants (e.g., colloidal silica, corn starch and talc); tablet lubricants (e.g., calcium stearate, magnesium stearate, mineral oil, stearic acid and zinc stearate); tablet/capsule opaquants (e.g., titanium dioxide); tablet polishing agents (e.g., carnuba wax and white wax); thickening agents (e.g., beeswax, cetyl alcohol and paraffin); tonicity agents (e.g., dextrose and sodium chloride); viscosity increasing agents (e.g., alginic acid, bentonite, carbomers, carboxymethylcellulose sodium, methylcellulose, povidone, sodium alginate and tragacanth); and wetting agents (e.g., heptadecaethylene oxycetanol, lecithins, polyethylene sorbitol monooleate, polyoxyethylene sorbitol monooleate, and polyoxyethylene stearate).

The compounds identified by the methods described herein may be administered as the sole pharmaceutical agent or in combination with one or more other pharmaceutical agents where the combination causes no unacceptable adverse effects. For example, the compounds of this invention can be combined with known anti-obesity, or with known anti-diabetic or other indication agents, and the like, as well as with admixtures and combinations thereof.

The compounds identified by the methods described herein may also be utilized, in free base form or in compositions, in research and diagnostics, or as analytical reference standards, and the like. Therefore, the present invention includes compositions which are comprised of an inert carrier and an effective amount of a compound identified by the methods described herein, or a salt or ester thereof. An inert carrier is any material which does not interact with the compound to be carried and which lends support, means of conveyance, bulk, traceable material, and the like to the compound to be carried. An effective amount of compound is that amount which produces a result or exerts an influence on the particular procedure being performed.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000)

The following examples are presented to illustrate the invention described herein, but should not be construed as limiting the scope of the invention in any way.

Capsule Formulation
A capsule formula is prepared from:

| | |
|---|---|
| A compound identified by the method | 40 mg |
| of this invention | |
| Starch | 109 mg |
| Magnesium stearate | 1 mg |

The components are blended, passed through an appropriate mesh sieve, and filled into hard gelatin capsules.

Tablet Formulation
A tablet is prepared from:

| | |
|---|---|
| A compound identified by the method | 25 mg |
| of this invention | |
| Cellulose, microcrystaline | 200 mg |
| Colloidal silicon dioxide | 10 mg |
| Stearic acid | 5.0 mg |

The ingredients are mixed and compressed to form tablets. Appropriate aqueous and non-aqueous coatings may be applied to increase palatability, improve elegance and stability or delay absorption.

Sterile IV Solution

A 5 mg/ml solution of the desired compound of this invention is made using sterile, injectable water, and the pH is adjusted if necessary. The solution is diluted for administration to 1-2 mg/ml with sterile 5% dextrose and is administered as an IV infusion over 60 minutes.

Intramuscular suspension
The following intramuscular suspension is prepared:

| | |
|---|---|
| A compound identified by the method | 50 mg/ml |
| of this invention | |
| Sodium carboxymethylcellulose | 5 mg/ml |
| TWEEN 80 | 4 mg/ml |
| Sodium chloride | 9 mg/ml |
| Benzyl alcohol | 9 mg/ml |

The suspension is administered intramuscularly.

Hard Shell Capsules

A large number of unit capsules are prepared by filling standard two-piece hard galantine capsules each with 100 mg of powdered active ingredient, 150 mg of lactose, 50 mg of cellulose and 6 mg of magnesium stearate.

Soft Gelatin Capsules

A mixture of active ingredient in a digestible oil such as soybean oil, cottonseed oil or olive oil is prepared and injected by means of a positive displacement pump into molten gelatin to form soft gelatin capsules containing 100 mg of the active ingredient. The capsules are washed and dried. The active ingredient can be dissolved in a mixture of polyethylene glycol, glycerin and sorbitol to prepare a water miscible medicine mix.

Immediate Release Tablets/Capsules

These are solid oral dosage forms made by conventional and novel processes. These units are taken orally without water for immediate dissolution and delivery of the medication. The active ingredient is mixed in a liquid containing ingredient such as sugar, gelatin, pectin and sweeteners. These liquids are solidified into solid tablets or caplets by freeze drying and solid state extraction techniques. The drug compounds may be compressed with viscoelastic and thermoelastic sugars and polymers or effervescent components to produce porous matrices intended for immediate release, without the need of water.

### EXAMPLES

The present invention is further illustrated by the following examples which should not be construed as limiting in any way. The contents of all cited references (including literature references, issued patents, published patent applications, and co-pending patent applications) cited throughout this application are hereby expressly incorporated by reference.

The Examples described below provide methods which can be used to identify test compounds which may act as agonists or antagonists of GPCR 192. Compounds identified by these methods may then be used for the treatment of central nervous system disorders, such as pain, metabolic disorders such as diabetes and obesity, immune disorders, and cancer. In addition, the methods below can be used to generate antibodies for PROK1, PROK2, or GPCR 192, which affect ligand binding and thus, receptor activity.

### Example 1

### Expression of the GPCR 192 cDNA in Reporter Cells and Assay of Activity

The cDNA for GPCR 192 (SEQ ID NO: 1) was inserted into pcDNA3.1 (Invitrogen, Carlsbad, Calif.). The cDNA for mouse GPR73 (SEQ ID NO: 7) was also inserted into pcDNA3.1. These constructs were stably transfected into a derivative of the CHO cell line that stably expresses human Gα16 and a luciferase reporter construct containing cyclic AMP response elements (CREs) upstream. The GPCR 192 reporter cell line was then used to determine agonist activities. Modulation of GPCR 192 activity is measured by determining changes in intracellular luciferase enzyme activity by standard methods (see, e.g., Ausubel et al., Current Protocols in Molecular Biology, John Wiley & Sons, 1988).

GPCR 192-expressing CHO reporter cells were plated in a 96-well plate 48 hours prior to the assay. Cell density at the time of the assay was approximately 5×10⁴/well. Cells were treated with the various agents (extract fractions, tissue culture supernatants, recombinant proteins, test compounds) for five hours. The culture medium was then removed and the cells were lysed with 1× lysis buffer (25 mM Tris-Pi, 2 mM CDTA, 10% glycerol, 1% Triton X-100, 0.03% DTT). The substrate solution (25 mM Tricine-KOH pH 7.8, 1 mM NaHCO₃, 2.5 mM MgSO₄, 0.1 mM EDTA, 450 μM Luciferin, 250 mM Coenzyme A, 500 μM ATP, 20 mM DTI) was then added and the plates were read immediately with a Wallac 1450 Microbeta Jet Liquid Scintillation and luminescence Counter (Perkin Elmer, Boston, Mass.).

### Example 2

### Purification of GPCR 192 Ligand from Bovine Hypothalmus

GPCR 192-stimulating activity was detected in the extracts from bovine hypothalmus. Ten kilograms (kg) of frozen bovine hypothalamus (Pel Freeze, Rogers, Ariz.) was homogenized in 80% acetone, 1 M acetic acid, 20 mM HCl (2 L/kg) using a Waring blender. The homogenate was then centrifuged at 30,000×g and the supernatant was retained. The supernatant was extracted 3× with an equal volume of diethyl ether (JT Baker, Phillipsburg, N.J.), retaining the aqueous phase. After removing residual ether by applying a constant stream of nitrogen for 2 hours, the aqueous material was adjusted to 20% acetonitrile and applied to two separate 450 ml SP Sepharose Fast Flow columns (Uppsala, Sweden) equilibrated in 6 mM HCl and 20% acetonitrile. The columns were then washed and a 0.1 M NaCl gradient applied. Fractions were then collected and tested for activity in GPCR 192-expressing CHO reporter cells expressing a luciferase reporter gene. Activity was determined by measuring the increase in expression of the luciferase reporter gene. After each chromatographic step, fractions were tested for GPCR 192-stimulating activity in the CHO-luciferase reporter assay. Active fractions were then used in the next chromatographic step.

Active fractions were then applied to a 10 g C18 Sep Pak (Waters, Milford, Mass.) equilibrated with 0.1% TFA. Each Sep Pak was washed with 5% acetonitrile/0.1% TFA, and then eluted with 50% acetonitrile/0.1% TFA. The eluant was flash frozen in liquid nitrogen and lyophilized. Lyophilized active fractions were reconstituted in 6 mM HCl and run on a 21.5×150 mm TosoHaas SP-5PW cation exchange prep column (Montgomeryville, Pa.) using a Beckman 125P prep HPLC system (Fullerton, Calif.). The column was washed with 450 mM NaCl and activity was eluted with 650 mM NaCl. The 650 mM NaCl eluate was loaded onto a TosoHaas 21.5×150 mm Phenyl-5PW RP prep column and eluted using a 6 mM HCl/acetonitrile gradient. Active fractions were then pooled and chromatographed on a 10×250 mm Vydac C 18 reversed phase column (Hisperia, Calif.) and eluted with a 6 mM HCl/acetonitrile gradient. Active fractions were then pooled and run on a Vydac diphenyl 1×50 mm microbore column using a Micro-Tech Scientific Ultra Plus II microbore HPLC system (Sunnyvale, Calif.). The column was run at 45° C. using a 6 mM HCl/acetonitrile gradient. Active fractions from this step were pooled. At this stage, a small portion of the active pool was analyzed by Edman degradation to determine the amino terminal sequence to the protein. The remainder was run on a PolyLC PolyCat A cation exchange 1×50 mm column (Columbia, Md.) in 10 mM Tris, pH 7.0 using a NaCl gradient. Active fractions were then separated on a Higgins Analytical HAISIL C8 column (Mountain View, Calif.) using a 6 mM HCl/acetonitrile gradient. The active fraction was then analyzed by mass spectroscopy.

### Example 3

### Protein Sequence Analysis

Amino-Terminal Sequence Analysis of the Purified Protein

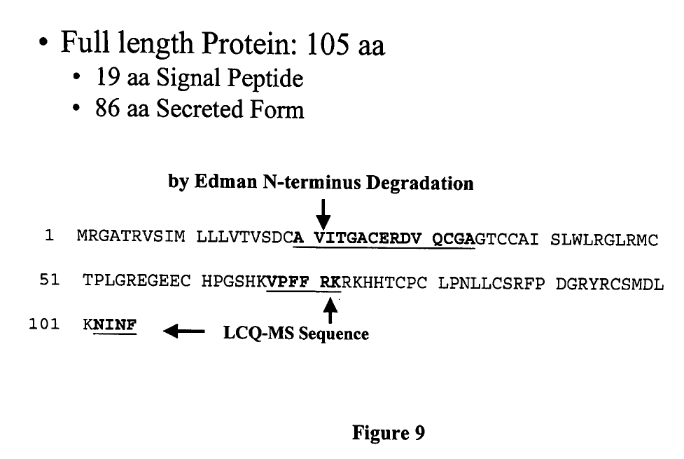

Automated Edman degradation of the enriched protein fraction and detection of phenylthiohydantoin-derivatives was performed on an Applied Biosystems Procise 494HT protein sequencing system (Foster City, Calif.) using the pulsed-liquid method according to manufacturer instructions. The amino-terminal sequence was determined to be: AVITGACERDVQCGA (SEQ ID NO: 9) (FIG. 9).

On-Line Capillary Liquid Chromatography-Electrospray Ionization-Tandem Mass Spectrometry (LC-MS/MS) Analysis

Proteins were digested with trypsin (Promega, Madison, Wis.) or lysyl endopeptidase (Wako, Richmond, Va.) overnight at 37° C. Resulting peptides were concentrated prior to mass spectrometric analysis. Mass spectrometric analysis was performed on a ThermoQuest Finnigan LCQ-DECA instrument (San Jose, Calif.). The peptide sequence was determined by on-line capillary liquid chromatography-electrospray ionization-tandem mass spectrometry (LC-MS/MS) analysis using a Dionex Vydac 300 um inner diameter C18 column (San Francisco, Calif.). An acetonitrile gradient was developed using a Hewlett-Packard 1100 pump operating at 0.4 ml/min, and the flow was split before the injector such that the flow rate through the column was 3 μl/min. The mass spectrometer was operated in data-dependent MS/MS mode. The following peptide sequences were obtained: VPFFRK (SEQ ID NO: 10) and NINF (SEQ ID NO: 11) (FIG. 9).

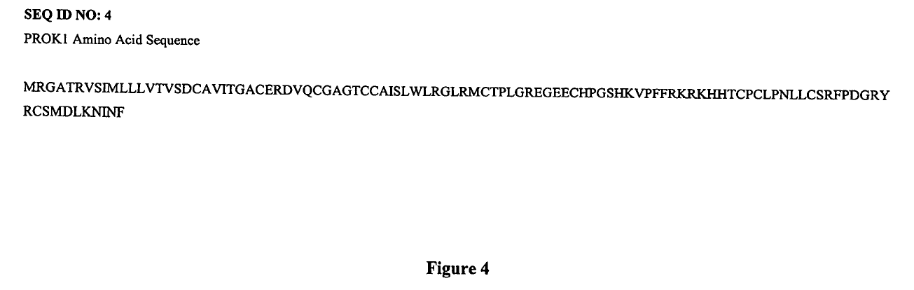

The peptide sequence information that was generated by Edman degradation and LC-MS/MS was used to determine if the purified protein was identical or related to known protein sequences by using the FINDPATTERNS and FASTA programs (Accelrys, San Diego, Calif.) to search non-redundant protein databases (e.g., SWISSPROT). By performing these analyses, the peptide sequences were identified as fragments of prokineticin 1 (GenBank accession number: AF333024; FIG. 4).

### Example 4

### Isolation of PROK1 and PROK2 cDNA

Oligonucleotides suitable for polymerase chain reaction (PCR) were designed to the coding sequence of PROK1 and PROK2. The oligonucleotide sequences were:

PROK1 Cloning Primers:

(SEQ ID NO: 12)
Forward:
5′-TTTGGATCCACCATGAGAGGTGCCACGCGAGTCTCA-3′
(SEQ ID NO: 13)
Reverse:
5′-TTTGCGGCCGCCTAATGGTGATGGTGATGGTGAAAATTGATGTTCTTCAAGTCCAT-3′

PROK2 Cloning Primers:

(SEQ ID NO: 14)
Forward:
5′-TTTGGATCCACCATGAGGAGCCTGTGCTGCGCCCCA-3′
(SEQ ID NO: 15)
Reverse:
5′-TTTGCGGCCGCTTAATGGTGATGGTGATGGTGCTTTTGGGCTAAACAAATAAATCG-3′

The forward oligonucleotides encode BamH I restriction endonuclease sites to facilitate cloning into the mammalian expression vector pcDNA3.1 (Invitrogen, Carlsbad, Calif.). The reverse oligonucleotides contain sequences encoding a 6× histidine tag at the carboxyl terminus of the protein prior to the stop codon to facilitate subsequent expression analysis and purification of PROK2. The reverse oligonucleotides also encode Not I restriction endonuclease sites for cloning purposes. Both the PROK1 and PROK2 cDNAs were isolated by PCR from human placenta cDNA, inserted into the pcDNA3.1 vector, and sequenced by the dideoxoy method methods (see, e.g., Ausubel et al., Current Protocols in Molecular Biology, John Wiley & Sons, 1988).

### Example 5

### Expression and Activity Assay of Recombinant PROK1 and PROK2

Baculovirus Expression

The cDNA for PROK1 or PROK2 with a histidine tag was inserted into pFastBac-1 (Invitrogen, Carlsbad, Calif.). The recombinant baculovirus expressing PROK2 was generated using the Bac-to-Bac system according to the manufacturer's protocol (Invitrogen, Carlsbad, Calif.). Sf9 cells were infected at an MOI of 5. Forty-eight (48) hours post-infection, the media was harvested.

Recombinant PROK2 was purified by Ni chelate chromatography by standard methods (see, e.g., Ausubel et al., 1988).

Mammalian Expression

PROK1 and PROK2 was also expressed transiently in 293T human embryonic kidney cells. The pcDNA3.1 PROK vectors or an empty vector control was transfected into 293T cells with Lipofectamine according to the manufacturer's protocol (Invitrogen, Carlsbad, Calif.). Twenty-four hours post-transfection, the media was replaced with fresh media containing 0.5% FBS. The tissue culture supernatants were harvested 24 hours later and tested on GPCR 192-expressing CHO reporter cells for the ability to induce intracellular luciferase activity.

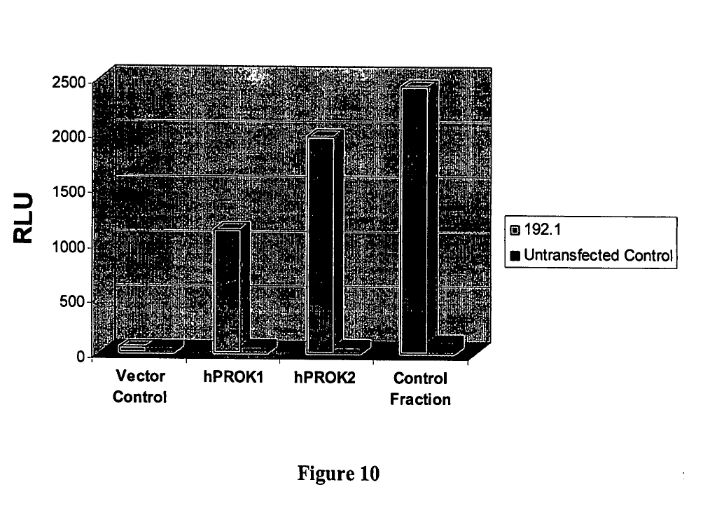

Tissue culture supernatants from 293T cells expressing PROK1 or PROK2 were found to activate both GPCR 192. However, supernatants form 293T cells transiently transfected with empty vector did not activate GPCR 192. Neither the empty vector nor PROK1 or PROK2 supenatants induced luciferase actvity in cells that did not express GPCR 192. Results are shown in FIG. 10.

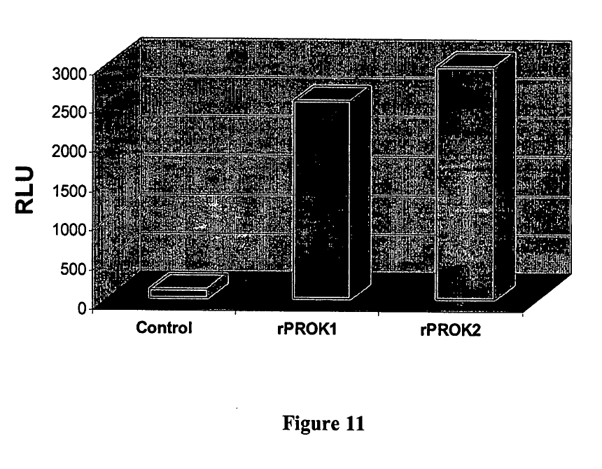

Recombinant baculoviruses expressing PROK1 (rPROK1), PROK2 (rPROK2), or an unrelated, control protein were used to infect Sf9 cells. The recombinant proteins were purified and then used to treat CHO reporter cells expressing GPCR192. Both rPROK1 and rPROK2 were capable of activating GPCR 192, but the control recombinant protein did not. Together, these results indicate that both PROK1 and PROK2 can act as ligands for GPCR192 (FIG. 11).

### Example 6

### GPCR 192 Expression Analysis

Multiple Tissue Array

To examine the expression pattern of GPCR 192, a multiple tissue expression array (Clontech, Palo Alto, Calif.) was probed. The GPCR 192 cDNA fragment was isolated and labeled with ³²P using the RediPrime kit (Amersham, Piscataway, N.J.). The probe was then hybridized to the array according to the manufacturer's protocol (Clontech, Palo Alto, Calif.). The data were visualized with a Storm 860 Optical Scanner (Molecular Dynamics, Sunnyvale, Calif.).

No detectable expression was observed for the majority of tissues on the Multiple Tissue Array. Expression was detected primarily in tissues of the central nervous system (CNS), including whole brain, fetal brain, parietal lobe, cerebellum, amygdala, caudate nucleus, thalamus, and spinal cord. Other tissues for which there was detectable expression included testis, bone marrow, and pancreas.

Quantitative PCR Expression Analysis

RNA Extraction and cDNA Preparation

Total RNA for TaqMan quantitative analysis was either purchased (Clontech, Palo Alto, Calif.) or extracted from tissues using TRIzol reagent (Life Technologies, Gaithersburg, Md.) according to a modified vendor protocol which utilizes the RNeasy protocol (Qiagen, Valencia, Calif.).

RNA (100 μg) was treated with DNase I using RNase free-DNase (Qiagen, Valencia, Calif.).

After elution and quantitation with Ribogreen (Molecular Probes, Inc., Eugene, Oreg.), each sample was reverse transcribed using the GibcoBRL Superscript II First Strand Synthesis System for RT-PCR according to vendor protocol (Life Technologies, Gaithersburg, Md.). The final concentration of RNA in the reaction mix was 50 ng/μL.

TaqMan Quantitative Analysis

Specific primers and probes were designed according to PE Applied Biosystems recommendations and are listed below:

(SEQ ID NO: 16)

forward primer:
5′-(CACCCAACTTTAATCCACCCC)-3′

(SEQ ID NO: 17)

reverse primer:
5′-(GGTCCGGGTCTTGGTCATG)-3′

(SEQ ID NO: 18)

probe:
5′-(FAM)-(CTTCAGTTATGGTGATTATGACCTCCCTATGGATG)
(TAMRA)-3′

where FAM=6-carboxy-fluorescein and TAMRA=6-carboxy-tetramethyl-rhodamine. The expected length of the PCR product was 112 bp.

Quantitation experiments were performed on reverse transcribed RNA (25 ng) from each sample. Each determination was done in duplicate. Ribosomal RNA (18S) was measured as a control using the Pre-Developed TaqMan Assay Reagents (PDAR) (PE Applied Biosystems, Foster City, Calif.). The assay reaction mix was as follows:

| | final | |
|---|---|---|
| TaqMan Universal PCR Master Mix (2x) | 1x | |
| PDAR control - 18S RNA (20x) | 1x | |
| Forward primer | 300 | nM |
| Reverse primer | 300 | nM |
| Probe | 200 | nM |
| cDNA | 25 | ng |
| Water | to 25 | μL |

PCR Conditions:
One cycle: 2 minutes at 50° C.
   10 minutes at 95° C.
40 cycles: 15 seconds at 95° C.
   1 minute at 60° C.

The experiment was performed on an ABI Prism 7700 Sequence Detector (PE Applied Biosystems, Foster City, Calif.). At the end of the run, fluorescence data acquired during PCR were processed as described in the ABI Prism 7700 user's manual. Fold change was calculated using the delta-delta CT method with normalization to the 18S values. Relative expression was calculated by normalizing to 18S (Δ Ct) and the data were then represented as relative expression based on the adjusted values. Relative expression of GPCR 192 in human central nervous system and other tissues is indicated in the following table.

| GPCR 192: Brain | | GPCR 192 Expression: | |
|---|---|---|---|
| Expression Profile | | Normal Tissues | |
| | 192 Relative | | 192 Relative |
| Sample Name | Expression | Sample Name | Expression |
| adrenal | − | adipose mes | − |
| adrenal cortex | − | adipose subq | + |
| adrenal gland | + | bone marrow | ++ |
| adrenal medulla | − | breast | − |
| amygdala | − | cecum | + |
| brain (whole) | ++ | colon | − |
| caudate | ++ | colon (ascending) | − |
| cerebellum | − | colon (descending) | − |
| cerebral cortex | ++ | colon (transverse) | − |
| corpus callosum | + | fetal liver | + |
| cortex | ++ | heart | − |
| fetal brain | +++ | ileum | − |
| hypothalamus | +/− | islets | − |
| medulla | ++ | kidney | − |
| pons | − | liver | − |
| prefrontal cort. | ++ | lung | − |
| putamen | +/− | ovary | + |
| spinal cord | + | pancreas | − |
| sub nigria | + | placenta | − |
| thalamus | +/− | prostate | − |
| NTC | − | salivary gland | + |
| | | skeletal muscle | − |
| | | small intestine | + |
| | | spleen | − |
| | | stomach | − |
| | | testes | ++ |
| | | thymus | − |
| | | thyroid | − |
| | | trachea | + |
| | | uterus | + |
| | | NTC | |

### Example 7

### Receptor Binding Methods: Standard Binding Assays

The polynucleotide encoding the GPCR 192 polypeptide (SEQ ID NO: 1) is inserted into the expression vector pcDNA3.1, and the expression vector (pcDNA3.1-GPCR 192) is transfected into mammalian cells. Alternatively, a recombinant baculovirus expressing GPCR 192 may be generated, and then be used to infect insect cells. Cells lines that express endogenous GPCR 192 may also be identified as source of membranes expressing the receptor.

The GPCR 192-expressing cells are scraped from a culture flask into 5 ml of Tris-HCl, 5 mM EDTA, pH 7.5, and lysed by sonication. Cell lysates are centrifuged at 1000 rpm for 5 minutes at 4° C. The supernatant is centrifuged at 30,000×g for 20 minutes at 4° C. The pellet is suspended in binding buffer containing 50 mM Tris-HCl, 5 mM MgSO₄, 1 mM EDTA, 100 mM NaCl, pH 7.5, supplemented with 0.1% BSA, 2 μg/ml aprotinin, 0.5 mg/ml leupeptin, and 10 mg/ml phosphoramidon. Optimal membrane suspension dilutions, defined as the protein concentration required to bind less than 10% of an added radioligand (i.e., ¹²⁵I-labeled PROK1 or PROK2), are added to 96-well polypropylene microtiter plates containing radioligand, non-labeled ligand or test compound, and binding buffer to a final volume of 250 μL. In equilibrium saturation binding assays, membrane preparations are incubated in the presence of increasing concentrations (0.1 nM to 4 nM) of ¹²⁵I-labeled ligand.

Binding reaction mixtures are incubated for one hour at 25° C. The reaction is stopped by filtration through GF/B filters treated with 0.5% polyethyleneimine, using a cell harvester. Radioactivity is measured by scintillation counting. The data are analyzed with a computerized non-linear regression program. Non-specific binding is defined as the amount of radioactivity remaining after incubation of membrane protein in the presence of 100 nM of unlabeled ligand. Protein concentration is measured by the Bradford method using Bio-Rad Reagent, with bovine serum albumin as a standard. The activity of GPCR 192 protein comprising the amino acid sequence of SEQ ID NO: 2 is demonstrated.

Binding assays are carried out in a binding buffer containing 50 mM HEPES, pH 7.4, 0.5% BSA, and 5 mM MgCl₂. The standard assay for radioligand binding to membrane fragments containing GPCR 192 polypeptides is carried out as follows in 96-well microtiter plates (e.g., Dynatech Immulon 11 Removawell plates). Radioligand is diluted in binding buffer+PMSF/Bacitracin to the desired cpm per 50 μl, then 50 μl aliquots are added to the wells. For non-specific binding samples, 5 μl of 40 μM cold ligand also is added per well.

Binding is initiated by adding 150 μl per well of membrane diluted to the desired concentration (1-30 g membrane protein/well) in binding buffer+PMSF/Bacitracin. Plates are then covered with Linbro mylar plate sealers (Flow Labs) and placed on a Dynatech Microshaker 11. Binding is allowed to proceed at room temperature for 1-2 hours and is stopped by centrifuging the plate for 15 minutes at 2,000×g. The supernatants are decanted, and the membrane pellets are washed once by addition of 200 μl of ice cold binding buffer, brief shaking, and centrifugation. The individual wells are placed in 12×75 mm tubes and counted in an LKB Gammamaster counter (78% efficiency). Specific binding by this method is identical to that measured when free ligand is removed by rapid (3-5 seconds) filtration and washing on polyethyleneimine-coated glass fiber filters.

Three variations of the standard binding assay are also used. Competitive radioligand binding assays with a concentration range of cold ligand vs. ¹²⁵I-labeled ligand are carried out as described above with one modification. All dilutions of ligands being assayed are made in PMSF/Bacitracin solution. Samples of, for example, test compounds (5 μl each) are then added to each microtiter well. Membranes and radioligand are diluted in binding buffer without protease inhibitors. Radioligand is added and mixed with cold ligand, and then binding is initiated by addition of membranes.

Chemical cross-linking of radioligand with receptor may be done after a binding step identical to the standard assay. However, the wash step is done with binding buffer minus BSA to reduce the possibility of non-specific cross-linking of radioligand with BSA. For example, after the radioligand binding step, membrane pellets are resuspended in 200 μl per microtiter plate well of ice-cold binding buffer without BSA. Then, 5 μl of 4 mM N-5-azido-2-nitrobenzoyloxysuceinimide (ANB-NOS, Pierce) in DMSO is added to each well and mixed. The samples are incubated on ice and UV-irradiated for 10 minutes with a Mineralight R-52G lamp (UVP Inc., San Gabriel, Calif.) at a distance of 5-10 cm. The samples are then transferred to Eppendorf microfuge tubes, the membranes pelleted by centrifugation, supernatants removed, and membranes solubilized in Laemmli SDS sample buffer for polyacrylamide gel electrophoresis (PAGE). Radiolabeled proteins are visualized by autoradiography of the dried gels with Kodak XAR film and Dupont image intensifier screens.

Larger scale binding assays to obtain membrane pellets for studies on solubilization of receptor:ligand complex and for receptor purification are also carried out. These are identical to the standard assays except that: (a) binding is carried out in polypropylene tubes in volumes from 1-250 ml, (b) concentration of membrane protein is always 0.5 mg/ml, and (c) for receptor purification, BSA concentration in the binding buffer is reduced to 0.25%, and the wash step is done with binding buffer without BSA, which reduces BSA contamination of the purified receptor.

### Example 8

### Methods for Identifying Agents that Affect the GPCR 192:PROK2 Interaction

Radioligand Binding Assays

Membrane fractions from cells expressing GPCR 192 are prepared as described above. In equilibrium saturation binding assays, membrane preparations are incubated in the presence of increasing concentrations (0.1 nM to 4 nM) of ¹²⁵I-labeled ligand or test compound (specific activity 2200 Ci/mmol). The binding affinities of different test compounds are determined in equilibrium competition binding assays, using 0.1 nM ¹²⁵I-labeled ligand (e.g., ¹²⁵I-labeled PROK2) in the presence of multiple different concentrations of each test compound. Binding reaction mixtures are incubated for one hour at 25° C. The reaction is stopped by filtration through GF/B filters treated with 0.5% polyethylencimine, using a cell harvester. Radioactivity is measured by scintillation counting. The data are analyzed by a computerized, non-linear regression program. Non-specific binding is defined as the amount of radioactivity remaining after incubation of membrane protein in the presence of 100 nM of unlabeled ligand.

A test compound which decreases the radioactivity of membrane protein by at least 15% relative to radioactivity of membrane protein which was not incubated with a test compound is identified as a compound which binds to a human GPCR 192 polypeptide.

### Example 9

### Effect of a Test Compound on Human GPCR 192-Mediated Cyclic AMP Formation

Receptor-mediated induction or inhibition of cAMP formation can be assayed in cells that express human GPCR 192. Cells are plated in 96-well plates and incubated in Dulbecco's phosphate buffered saline (PBS) supplemented with 10 mM HEPES, 5 mM theophylline, 2 μg/ml aprotinin, 0.5 mg/ml leupeptin, and 10 μg/ml phosphoramidon for 20 minutes at 37° C. in 5% CO₂. A test compound is added and incubated for an additional 10 minutes at 37° C. Ligand is then added and incubated for an additional 10 minutes. The medium is aspirated, and the reaction is stopped by the addition of 100 mM HCl. The plates are stored at 4° C. for 15 minutes. cAMP content in the stopping solution is measured by radioimmunoassay using a commercially available kit (e.g., Amersham, Piscataway, N.J.). Radioactivity is quantified using a gamma counter equipped with data reduction software. A test compound which decreases radioactivity of the contents of a well relative to radioactivity of the contents of a well in the absence of the test compound is identified as a potential enhancer of GPCR 192-dependent cAMP formation. A test compound which increases radioactivity of the contents of a well relative to radioactivity of the contents of a well in the absence of the test compound is identified as a potential inhibitor of GPCR 192-dependent cAMP formation.

### Example 10

### Effect of a Test Compound on Human GPCR 192-Induced Luciferase Activity in a Reporter Cell Line

A derivative of the CHO cell line that stably expresses human Ga 16 and a luciferase reporter construct containing cyclic AMP response elements (CREs) upstream and that also stably expresses GPCR 192 are seeded onto 96- or 384-well culture dishes and incubated overnight at 37° C. in a CO₂ incubator. Test compounds are added to the well and incubated at 37° C. in a CO₂ incubator for 30 minutes. PROK2 ligand is then added at approximately the concentration required to elicit 50-60% of the maximal response. The cells are then incubated for 4-6 hours at 37° C. in a CO₂ incubator. The media is then removed from the wells and the cells are lysed in 1× lysis buffer (25 mM Tris-Pi, 2 mM CDTA, 10% glycerol, 1% Triton X-100, 0.03% DTT). The substrate solution (25 mM Tricine-KOH pH 7.8, 1 mM NaHCO₃, 2.5 mM MgSO₄, 0.1 mM EDTA, 450 μM Luciferin, 250 mM Coenzyme A, 500 μM ATP, 20 mM DTT) is then added and the plates are read immediately on a Wallac 1450 Micobeta Trilux Plate Reader (Perkin Elmer, Boston, Mass.). Test compounds that decrease the amount of luciferase enzyme activity induced by the GPCR 192:PROK2 interaction are identified as inhibitors of GPCR 192-dependent luciferase induction. Test compounds that increase the amount of luciferase enzyme activity induced by the GPCR 192:PROK2 interaction are identified as enhancers of GPCR 192-dependent luciferase induction.

### Example 11

### Effect of a Test Compound on the Mobilization of Intracellular Calcium

Intracellular free calcium concentration can be measured with a fluorometric assay using the fluorescent indicator dye (e.g., Fluo-3, Molecular Probes, Eugene, Oreg.) using a FLIPR system according to the manufacturer's protocol (Molecular Devices, Sunnyvale, Calif.). Stably transfected cells are seeded onto 96- or 384-well culture dishes. Cells are washed with HBS, incubated with a test compound, incubated further with ligand PROK2. Fluorescence emission is determined at 510-570 nm, with excitation wavelengths at 488 nm. The data are analyzed with the manufacturer's software. A test compound that increases the fluorescence by at least 15% relative to fluorescence in the absence of a test compound is identified as a compound that mobilizes intracellular calcium in a GPCR 192-dependent manner. Test compounds that decrease the relative fluorescence are identified as inhibitors of GPCR 192-dependent intracellular calcium mobilization.

### Example 12

### Effect of a Test Compound on Phosphoinositide Metabolism

Cells that stably express human GPCR 192 cDNA are plated into 96-well plates and grown to confluence. The day before the assay, the growth medium is changed to 100 μl of medium containing 1% serum and 0.5 μCi ³H-myinositol (NEN, Perkin Elmer, Boston, Mass.). The plates are incubated overnight in a CO₂ incubator (5% CO₂, at 37° C.). Immediately before the assay, the medium is removed and replaced by 200 μl of PBS containing 10 mM LiCl, and the cells are equilibrated with the new medium for 20 minutes. During this interval, cells also are equilibrated with antagonist, added as a 10 μl aliquot of a 20-fold concentrated solution in PBS.

The ³H-inositol phosphate (IP) accumulation from inositol phospholipid metabolism is initiated by adding 10 μl of a solution containing a test compound. To the first well, 10 μl are added to measure basal accumulation. Eleven different concentrations of a test compound are assayed in the following 11 wells of each plate row. All assays are performed in duplicate by repeating the same additions in two consecutive plate rows.

The plates are incubated in a CO₂ incubator for one hour. The reaction is terminated by addition of 15 μl of 50% v/v trichloroacetic acid (TCA) and incubating at 4° C. for 40 minutes. After neutralizing TCA with 40 μl of 1 M Tris, the content of the wells is transferred to a Multiscreen HV filter plate (Millipore, Bedford, Mass.) containing Dowex AG1-X8 (200-400 mesh, formate form). The filter plates are prepared by adding 200 μl of Dowex AG1-X8 suspension (50% v/v, water:resin) to each well. The filter plates are placed on a vacuum manifold to wash or elute the resin bed. Each well is washed twice with 200 μl of water, followed by 2×200 μL washes with 5 mM sodium tetraborate/60 mM ammonium formate.

The ³H-IPs are eluted into empty 96-well plates with 200 μl of 1.2 M ammonium formate/0.1 M formic acid. The content of the wells is added to 3 ml of scintillation cocktail, and radioactivity is determined by liquid scintillation counting.

### Example 13

### Effect of a Test Compound on Receptor Desensitization/Internalization

Several methods may be used to measure the effects of test compounds on PROK2-induced internalization of GPCR 192 and/or the association of GPCR 192 with β-arrestin. PROK2 is tagged with a fluorescent label (e.g., fluorescein isothiocyanate) by conventional methods. Cells stably expressing GPCR 192 are seeded in a 96-well culture dish and incubated in a CO₂ incubator at 37° C. until they reach the desired density. The cells are then treated with the test compound and incubated in a CO₂ incubator at 37° C. for 30 minutes. The labeled PROK2 is then added. The effects on receptor internalization are then measured with an imaging apparatus capable of real time fluorescent imagining such as a Cellomics Array Scan. Test compounds that reduce the amount of fluorescent ligand that is internalized into the cells are identified as inhibitors of PROK2-induced GPCR 192 internalization. Test compounds that increase the amount of fluorescent ligand that is internalized into the cells are identified as enhancers of PROK2-induced GPCR 192:β-arrestin association.

Cells that stably coexpress GPCR 192 and β-arrestin fused to green fluorescent protein (GFP) are seeded in a 96-well culture dish and incubated in a CO₂ incubator at 37° C. until they reach the desired density (see, e.g., U.S. Pat. Nos. 5,891,646; 6,096,705; 6,110,693). The cells are then treated with the test compound and incubated in a CO₂ incubator at 37° C. for 30 minutes. The PROK2 ligand is then added. The effects on receptor internalization are observed as ligand-dependent localization of β-arrestin first to the cell membrane and then to intracellular vesicles. These effects are measured with an imaging platform capable of real time fluorescent imaging such as a Cellomics Array Scan. Test compounds that reduce the amount of receptor internalization into the cells are identified as inhibitors of PROK2-induced GPCR 192: β-arrestin association. Test compounds that increase the ratio of green light to blue light are identified as enhancers of PROK2-induced GPCR 192:β-arrestin association.

The BRET assay technology (Packard Bioscience, Meriden, Conn.) measures the fluorescent energy transfer that occurs when a GPCR, which is fused to luciferase, associates with a β-arrestin-GFP fusion protein upon activation of the receptor by a ligand. A vector that expresses GPCR 192 fused to the Renilla luciferase protein is stably transfected into cells that express a β-arrestin-GFP fusion protein. In the presence of a coelenterazine, the luciferase emits a blue light. When the luciferase is in close proximity to GFP, it causes the GFP to emit green light by fluorescence energy resonance transfer. Ligand-induced association of the receptor with β-arrestin is monitored by the change in the ratio of blue and green light emitted by the cells. The transfected cells are seeded in a 96- or 384-well culture dishes and incubated in a CO₂ incubator at 37° C. until they reach the desired density. The cells are then treated with the test compound and incubated in a CO₂ incubator at 37° C. for 30 minutes. The PROK2 ligand is then added and the cells are incubated for 10-30 minutes. The effects on receptor internalization are then measured with an imaging platform capable of reading two-color emission such as a Fusion Universal Microplate Analyzer (Packard Instruments). Test compounds that reduce the ratio of green light to blue light are identified as inhibitors of PROK2-GPCR 192: β-arrestin association. Test compounds that increase the ratio of green light to blue light are identified as enhancers of PROK2-induced GPCR 192: β-arrestin association.

### Example 14

### Preparation of Antibodies that Bind PROK1 or PROK2

This example illustrates preparation of monoclonal antibodies that can specifically bind PROK1 or PROK2. Techniques for producing the monoclonal antibodies are well known in the art and are described, for example, in Lane and Harlow (Antibodies, Cold Spring Harbor Press, 1988). A variety of immunogens may be employed including purified PROK1 or PROK2, fusion proteins containing PROK1 or PROK2, or cells expressing recombinant PROK1 or PROK2 on the cell surface. The immunogen may be selected by someone skilled in the art without undue experimentation.

Mice, such as Balb/c, are immunized with the PROK1 or PROK2 immunogen that has been emulsified in complete Freund's adjuvant and injected subcutaneously or intraperitoneally in an amount from 1-100 micrograms. Alternatively, the immunogen is emulsified in MPL-TDM adjuvant (Corixa Corporation, Seattle, Wash.) and injected into the animal's hind foot pads. The immunized mice are then boosted 10 to 12 days later with additional immunogen emulsified in the selected adjuvant. For several weeks thereafter, the mice may also be boosted with additional immunization injections. Periodically, serum samples are obtained from the mice by retro-orbital bleeding and tested in ELISA assays to detect anti-PROK1 or anti-PROK2 antibodies.

When a suitable antibody titer has been detected, animals producing anti-PROK1 or anti-PROK2 antibodies can be injected with a final intravenous injection of PROK1 or PROK2. The mice are then sacrificed three to four days later and the spleen cells are harvested. The spleen cells are then fused (using 35% polyethylene glycol) to a selected murine myeloma cell line (e.g., P3X63Ag8.653, ATCC, Manassas, Va.). This cell fusion generates hybridoma cells which can then be plated in 96-well tissue culture plates containing HAT (hypoxanthine, aminopterin, and thymidine) medium to inhibit proliferation of non-fused cells, myeloma hybrids, and spleen cell hybrids.

The hybridoma cells are screened in an ELISA for reactivity against PROK1 or PROK2. Determination of “positive” hybridoma cells secreting the desired monoclonal antibodies against PROK1 or PROK2 is within the skill in the art. The positive hybridoma cells can be injected intraperitoneally into syngeneic Balb/c mice to produce ascites containing the anti-PROK1 or anti-PROK2 monoclonal antibodies. Alternatively, the hybridoma cells can be grown in tissue culture flasks or roller bottles. Purification of the monoclonal antibodies produced in the ascites can be accomplished using ammonium sulfate precipitation, followed by gel exclusion chromatography. Alternatively, affinity chromatography based upon binding of antibody to protein A or protein G can be employed.

### Example 15

### Preparation of PROK1 and PROK2 Peptides

Synthetic peptides to PROK1 and PROK2 may be used to generate antibodies or as biologically active agents that regulate GPCR 192 activity. PROK1 and PROK2 peptides are synthesized on an Applied Biosystems (Foster City, Calif.) 433A peptide synthesizer using Fmoc (9-Fluorenylmethoxycarbonyl) chemistry with HBTU (2-(1H-benzotriazole-1-yl)-1,1,3,3-tetramethyluronium hexafluorophosphate) activation according to the manufacturer's protocol. All peptide synthesis reagents may be purchased from Applied Biosystems (Foster City, Calif.). The peptides are then cleaved and deprotected with 84.6% trifluoroacetic acid (Applied Biosystems), 4.4% thioanisole (Aldrich, Milwaukee, Wis.), 2.2% ethanedithiol (Aldrich), 4.4% liquified phenol (J.T. Baker, Phillipsburg, N.J.) and 4.4% water for 2 hours. The crude peptides are then filtered from the resin and precipitated with t-butyl methyl ether (Aldrich) and centrifuged. The pellets are washed in t-butyl methyl ether and centrifuged. The ether is removed from the pellets and the pellets are dissolved in 0.1% trifluoroacetic acid. Purification is performed using a Dynamx (Varian Analytical Instruments, Walnut Creek, Calif.) C18 21.5×250 mm prep reversed-phase HPLC column using a Beckman 125P prep HPLC system.

It should be apparent to one of ordinary skill in the art that changes and modifications can be made to this invention without departing from the spirit or scope of the invention as it is set forth herein.

We claim:

1. A method for identifying an agonist or antagonist of GPCR 192, comprising the steps of:
contacting GPCR 192 with a test compound; and
detecting agonist or antagonist activity.

2. The method of claim 1, wherein said method further comprises PROK1 or PROK2, or variants or derivatives thereof; and detecting agonist or antagonist activity of the test compound in the presence of PROK1 or PROK2 or variants thereof.

3. The method of claim 2, wherein the step of contacting is in a cell.

4. The method of claim 3, wherein said cell is mammalian.

5. The method of claim 4, wherein said cell is in vivo.

6. The method of claim 4, wherein said cell is in vitro.

7. The method of claim 1, wherein the step of contacting is in a cell-free system.

8. The method of claim 1, wherein the test compound comprises a detectable label.

9. The method of claim 2, wherein PROK1 or PROK2 comprises a detectable label.

10. The method of claim 1, wherein GPCR192 comprises the amino acid sequence shown in SEQ ID NO:2.

11. The method of claim 2, wherein PROK1 comprises the amino acid sequence shown in SEQ ID NO:4.

12. The method of claim 2, wherein PROK2 comprises the amino acid sequence shown in SEQ ID NO:6.

13. The method of claim 1, wherein said method further comprises a reporter gene whose expression is modulated by GPCR 192; and said GPCR 192 activity is measured by the expression level of said reporter gene.

14. A method of treating central nervous system disorders, metabolic disorders, immune disorders, or cancer comprising administering to a patient in need thereof an effective amount of a compound identified by the method of claim 1.

15. A method of treating central nervous system disorders, metabolic disorders, immune disorders, or cancer comprising administering to a patient in need thereof an effective amount of a compound identified by the method of claim 2.

16. A pharmaceutical composition comprising an effective amount of a compound identified by the method of claim 1 in combination with a pharmaceutically acceptable carrier.

17. A pharmaceutical composition comprising an effective amount of a compound identified by the method of claim 2 in combination with a pharmaceutically acceptable carrier.

18. A pharmaceutical composition comprising an antibody that specifically binds to PROK1 or PROK2, and a pharmaceutically acceptable carrier.

19. A pharmaceutical composition comprising an antisense oligonucleotide that hybridizes to a polynucleotide encoding PROK1 or PROK2 and reduces expression of the polynucleotide; and a pharmaceutically acceptable carrier.

20. A pharmaceutical composition comprising an antisense oligonucleotide that hybridizes to a polynucleotide encoding GPCR 192 and reduces expression of the polynucleotide; and a pharmaceutically acceptable carrier.

21. A pharmaceutical composition comprising an antibody that specifically binds to GPCR 192, and a pharmaceutically acceptable carrier.

22. Use of a reagent that modulates the activity of PROK1, PROK2, or GPCR192 in the manufacture of a medicament for treating central nervous system disorders, metabolic disorders, immune disorders, or cancer.

23. Use of an antibody that specifically binds to PROK1, PROK2, or GPCR192 and modulates the activity of PROK1, PROK2, or GPCR192 in the manufacture of a medicament for treating central nervous system disorders, metabolic disorders, immune disorders, or cancer.

24. Use of an oligonucleotide that hybridizes to a polynucleotide encoding PROK1, PROK2, or GPCR192 and alters the expression of the polynucleotide in the manufacture of a medicament for treating central nervous system disorders, metabolic disorders, immune disorders, or cancer.


SEQUENCE LISTING (18 sequences)

SEQ ID NO: 1 - DNA, 1155 bp, Homo sapiens
atggcagcccagaatggaaacaccagtttcacacccaactttaatccaccccaagaccatgcctcctccctctcctttaacttcagttatggtgattatgacctccctatggatgaggatgaggacatgaccaagacccggaccttcttcgcagccaagatcgtcattggcattgcactggcaggcatcatgctggtctgcggcatcggtaactttgtctttatcgctgccctcacccgctataagaagttgcgcaacctcaccaatctgctcattgccaacctggccatctccgacttcctggtggccatcatctgctgccccttcgagatggactactacgtggtacggcagctctcctgggagcatggccacgtgctctgtgcctccgtcaactacctgcgcaccgtctccctctacgtctccaccaatgccttgctggccattgccattgacagatatctcgccatcgttcaccccttgaaaccacggatgaattatcaaacggcctccttcctgatcgccttggtctggatggtgtccattctcattgccatcccatcggcttactttgcaacagaaaccgtcctctttattgtcaagagccaggagaagatcttctgtggccagatctggcctgtggatcagcagctctactacaagtcctacttcctcttcatctttggtgtcgagttcgtgggccctgtggtcaccatgaccctgtgctatgccaggatctcccgggagctctggttcaaggcagtccctgggttccagacggagcagattcgcaagcggctgcgctgccgcaggaagacggtcctggtgctcatgtgcattctcacggcctatgtgctgtgctgggcacccttctacggtttcaccatcgttcgtgacttcttccccactgtgttcgtgaaggaaaagcactacctcactgccttctacgtggtcgagtgcatcgccatgagcaacagcatgatcaacaccgtgtgcttcgtgacggtcaagaacaacaccatgaagtacttcaagaagatgatgctgctgcactggcgtccctcccagcgggggagcaagtccagtgctgaccttgacctcagaaccaacggggtgcccaccacagaagaagtggactgtatcaggctgaagtga

SEQ ID NO: 2 - protein, 384 aa, Homo sapiens
MAAQNGNTSFTPNFNPPQDHASSLSFNFSYGDYDLPMDEDEDMTKTRTFFAAKIVIGIALAGIMLVCGIGNFVFIAALTRYKKLRNLTNLLIANLAISDFLVAIICCPFEMDYYVVRQLSWEHGHVLCASVNYLRTVSLYVSTNALLAIAIDRYLAIVHPLKPRMNYQTASFLIALVWMVSILIAIPSAYFATETVLFIVKSQEKIFCGQIWPVDQQLYYKSYFLFIFGVEFVGPVVTMTLCYARISRELWFKAVPGFQTEQIRKRLRCRRKTVLVLMCILTAYVLCWAPFYGFTIVRDFFPTVFVKEKHYLTAFYVVECIAMSNSMINTVCFVTVKNNTMKYFKKMMLLHWRPSQRGSKSSADLDLRTNGVPTTEEVDCIRLK

SEQ ID NO: 3 - DNA, 1377 bp, Homo sapiens
ggggaagcgagaggcatctaagcaggcagtgttttgccttcaccccaagtgaccatgagaggtgccacgcgagtctcaatcatgctcctcctagtaactgtgtctgactgtgctgtgatcacaggggcctgtgagcgggatgtccagtgtggggcaggcacctgctgtgccatcagcctgtggcttcgagggctgcggatgtgcaccccgctggggcgggaaggcgaggagtgccaccccggcagccacaaggtccccttcttcaggaaacgcaagcaccacacctgtccttgcttgcccaacctgctgtgctccaggttcccggacggcaggtaccgctgctccatggacttgaagaacatcaatttttaggcgcttgcctggtctcaggatacccaccatccttttctgagcacagcctggatttttatttctgccatgaaacccagctcccatgactctcccagtccctacactgactaccctgatctctcttgtctagtacgcacatatgcacacaggcagacatacctcccatcatgacatggtccccaggctggcctgaggatgtcacagcttgaggctgtggtgtgaaaggtggccagcctggttctcttccctgctcaggctgccagagaggtggtaaatggcagaaaggacattccccctcccctccccaggtgacctgctctctttcctgggccctgcccctctccccacatgtatccctcggtctgaattagacattcctgggcacaggctcttgggtgcattgctcagagtcccaggtcctggcctgaccctcaggcccttcacgtgaggtctgtgaggaccaatttgtgggtagttcatcttccctcgattggttaactccttagtttcagaccacagactcaagattggctcttcccagagggcagcagacagtcaccccaaggcaggtgtagggagcccagggaggccaatcagccccctgaagactctggtcccagtcagcctgtggcttgtggcctgtgacctgtgaccttctgccagaattgtcatgcctctgaggccccctcttaccacactttaccagttaaccactgaagcccccaattcccacagcttttccattaaaatgcaaatggtggtggttcaatctaatctgatattgacatattagaaggcaattagggtgtttccttaaacaactcctttccaaggatcagccctgagagcaggttggtgactttgaggagggcagtcctctgtccagattggggtgggagcaagggacagggagcagggcaggggctgaaaggggcactgattcagaccagggaggcaactacacaccaacctgctggctttagaataaaagcaccaactgaactg

SEQ ID NO: 4 - protein, 105 aa, Homo sapiens
MRGATRVSIMLLLVTVSDCAVITGACERDVQCGAGTCCAISLWLRGLRMCTPLGREGEECHPGSHKVPFFRKRKHHTCPCLPNLLCSRFPDGRYRCSMDLKNINF

SEQ ID NO: 5 - DNA, 1406 bp, Homo sapiens
gagggcgccatgaggagcctgtgctgcgccccactcctgctcctcttgctgctgccgccgctgctgctcacgccccgcgctggggacgccgccgtgatcaccggggcttgtgacaaggactcccaatgtggtggaggcatgtgctgtgctgtcagtatctgggtcaagagcataaggatttgcacacctatgggcaaactgggagacagctgccatccactgactcgtaaagttccattttttgggcggaggatgcatcacacttgcccatgtctgccaggcttggcctgtttacggacttcatttaaccgatttatttgtttagcccaaaagtaatcgctctggagtagaaaccaaatgtgaatagccacatcttacctgtaaagtcttacttgtgattgtgccaaacaaaaaatgtgccagaaagaaatgctcttgcttcctcaactttccaagtaacatttttatctttgatttgtaaatgatttttttttttttttttatcgaaagagaattttacttttggatagaaatatgaagtgtaaggcattatggaactggttcttatttccctgtttgtgttttggtttgatttggcttttttcttaaatgtcaaaaacgtacccattttcacaaaaatgaggaaaataagaatttgatattttgttagaaaaacttttttttttttttctcaccaccccaagccccatttgtgccctgccgcacaaatacacctacagcttttggtcccttgcctcttccacctcaaagaatttcaaggctcttaccttactttatttttgtccatttctcttccctcctcttgcattttaaagtggagggtttgtctctttgagtttgatggcagaatcactgatgggaatccagctttttgctggcatttaaatagtgaaaagagtgtatatgtgaacttgacactccaaactcctgtcatggcacggaagctaggagtgctgctggacccttcctaaacctgtcactcaagaggacttcagctctgctgttgggctggtgtgtggacagaaggaatggaaagccaaattaatttagtccagatttctaggtttgggtttttctaaaaataaaagattacatttacttcttttactttttataaagttttttttccttagtctcctacttagagatattctagaaaatgtcacttgaagaggaagtatttattttaatctggcacaacactaattaccatttttaaagcggtattaagttgtaatttaaaccttgtttgtaactgaaaggtcgattgtaatggattgccgtttgtacctgtatcagtattgctgtgtaaaaattctgtatcagaataataacagtactgtatatcatttgatttattttaatattatatccttatttttgtc

SEQ ID NO: 6 - protein, 108 aa, Homo sapiens
MRSLCCAPLLLLLLLPPLLLTPRAGDAAVITGACDKDSQCGGGMCCAVSIWVKSIRICTPMGKLGDSCHPLTRKVPFFGRRMHHTCPCLPGLACLRTSFNRFICLAQK

SEQ ID NO: 7 - DNA, 1182 bp, Mouse
atggagaccactgtcggggctctgggtgagaataccacagacaccttcaccgacttcttttctgcactcgatggccatgaagcccaaaccggctcgttaccattcactttcagctacggtgactatgacatgcccctggatgaagaggaagatgtgaccaattctcggactttctttgctgccaagattgtcattggcatggctttggtgggtatcatgctagtgtgtggcatcggcaacttcatctttatcactgccctggcccgctacaaaaagctccgcaacctcaccaacctgcttatcgccaacctggccatttcagacttcctcgtggccatcgtgtgctgcccctttgagatggactactatgtggtgcgccagctctcctgggagcatggtcatgtcctgtgcgcctctgtcaactacttgcgtaccgtctccctctacgtctccactaacgccctactggccattgccattgacaggtatctggccattgtgcacccgctgagaccgcggatgaagtgtcaaacagccgccggcctgatcttcctggtgtggtcagtatccatcctcatcgccattccagctgcctacttcaccactgagaccgtgctggtcatcgtggagagacaggagaagatcttctgtggtcagatctggccggtggatcagcagttctactacaggtcctatttccttttggttttcggcctcgagttcgtgggccccgtagtcgccatgaccttgtgctatgccagggtgtcccgggagctctggttcaaggcggtgccaggcttccagacagagcagatccgccggacggtgcgctgccgccgcaggacggtgctggggctcgtgtgcgtcctctctgcctatgtgctgtgctgggctcccttctatggcttcactatcgtgcgtgacttcttcccctccgtgtttgtgaaggagaagcactacctcaccgccttctatgtggtggagtgcatcgccatgagcaacagcatgatcaatacgctctgctttgtgactgtcaggaataacaccagtaagtacctcaagaggatcctgcggcttcagtggagggcctctcccagcgggagcaaggccagcgctgacctcgacctcaggaccacgggaatacctgccaccgaggaggtggactgcatccgactgaaataa

SEQ ID NO: 8 - protein, 393 aa, Mouse
METTVGALGENTTDTFTDFFSALDGHEAQTGSLPFTFSYGDYDMPLDEEEDVTNSRTFFAAKIVIGMALVGIMLVCGIGNFIFITALARYKKLRNLTNLLIANLAISDFLVAIVCCPFEMDYYVVRQLSWEHGHVLCASVNYLRTVSLYVSTNALLAIAIDRYLAIVHPLRPRMKCQTAAGLIFLVWSVSILIAIPAAYFTTETVLVIVERQEKIFCGQIWPVDQQFYYRSYFLLVFGLEFVGPVVAMTLCYARVSRELWFKAVPGFQTEQIRRTVRCRRRTVLGLVCVLSAYVLCWAPFYGFTIVRDFFPSVFVKEKHYLTAFYVVECIAMSNSMINTLCFVTVRNNTSKYLKRILRLQWRASPSGSKASADLDLRTTGIPATEEVDCIRLK

SEQ ID NO: 9 - protein, 15 aa, Homo sapiens
AVITGACERDVQCGA

SEQ ID NO: 10 - protein, 6 aa, Homo sapiens
VPFFRK

SEQ ID NO: 11 - protein, 4 aa, Homo sapiens
NINF

SEQ ID NO: 12 - DNA, 36 bp, Homo sapiens
tttggatccaccatgagaggtgccacgcgagtctca

SEQ ID NO: 13 - DNA, 56 bp, Homo sapiens
tttgcggccgcctaatggtgatggtgatggtgaaaattgatgttcttcaagtccat

SEQ ID NO: 14 - DNA, 36 bp, Homo sapiens
tttggatccaccatgaggagcctgtgctgcgcccca

SEQ ID NO: 15 - DNA, 56 bp, Homo sapiens
tttgcggccgcttaatggtgatggtgatggtgcttttgggctaaacaaataaatcg

SEQ ID NO: 16 - DNA, 21 bp, Homo sapiens
cacccaactttaatccacccc

SEQ ID NO: 17 - DNA, 19 bp, Homo sapiens
ggtccgggtcttggtcatg

SEQ ID NO: 18 - DNA, 35 bp, Homo sapiens
cttcagttatggtgattatgacctccctatggatg